(19) United States
(12) Patent Application Publication
(10) Pub. No.: US 2004/0161754 A1
(43) Pub. Date: Aug. 19, 2004

(54) Human erg2 potassium channel

(76) Inventors: Kimberly L. Folander, Bethlehem, PA (US); Yuan Liu, Lansdale, PA (US); Edward J. McKenna, Lansdale, PA (US); Richard J. Swanson, Lansdale, PA (US)

Correspondence Address: MERCK AND CO INC, P O BOX 2000, RAHWAY, NJ, 070650907

(21) Appl. No.: 10/432,171

(22) PCT Filed: Nov. 16, 2001
(86) PCT No.: PCT/US01/43490

(51) Int. Cl. C12Q 1/68; C07H 21/04; C12N 5/06; C07K 14/705

(52) U.S. Cl. 435/6; 435/69.1; 435/320.1; 435/325; 530/350; 536/23.5

(57) ABSTRACT

The present invention is directed to novel human DNA sequences encoding h-erg2 proteins, the protein encoded by the DNA sequences, vectors comprising the DNA sequences, host cells containing the vectors, and methods of identifying inhibitors and activators of potassium channels containing the h-erg2 proteins.

### CROSS-REFERENCE TO RELATED APPLICATIONS

This application claims the benefit of U.S. Provisional Application No. 60/249,981, filed Nov. 20, 2000, the contents of which are incorporated herein by reference in their entirety.

### STATEMENT REGARDING FEDERALLY-SPONSORED R&D

Not applicable.

### REFERENCE TO MICROFICHE APPENDIX

Not applicable.

### FIELD OF THE INVENTION

The present invention is directed to a novel human DNA sequence encoding a potassium channel subunit, protein encoded by the DNA sequence, methods of expressing the protein in recombinant cells, and methods of identifying activators and inhibitors of potassium channels comprising the subunit.

### BACKGROUND OF THE INVENTION

Potassium channels are ion channels found in nearly all cells where they form transmembrane pores that selectively allow potassium ions to pass through the membrane. Potassium channels can be classified into various types based on their molecular architecture, their biophysical and pharmacologic properties, and the functions they perform.

Different types of potassium channels may preferentially conduct potassium currents in either the inward or outward direction and these different types of channels perform distinct functions in the cells and tissues in which they are expressed. For example, classical inward rectifier potassium channels preferentially conduct potassium currents into the cell at voltages negative to the potassium equilibrium potential (E_{K}) and thereby largely function to stabilize cellular membrane potentials near the equilibrium potential for potassium ions. In contrast, delayed rectifier potassium channels are typically closed until the membrane is depolarized to the channel's activation threshold and then delayed rectifiers preferentially conduct potassium currents out of the cell at these depolarized potentials. Delayed rectifiers contribute to the repolarization of the membrane following cellular excitation. Both classes are important in regulating the excitability of certain cardiovascular and neurological cells and tissues.

The ether-a-go-go (eag) family of potassium channels is a group of potassium channels whose first discovered member, eag, was identified in Drosophila due to its mutant leg shaking phenotype (Kaplan & Trout, 1969, Genetics 61:399). When cloned, eag was found to have structural features typical of delayed rectifier potassium channels and expression studies in Xenopus oocytes confirmed that eag was a functional potassium channel, indeed producing a delayed rectifier (Warmke et al., 1991, Science 252:1560-1562; Robertson et al., 1993, Biophys. J. Abstr. 64:A340; Bruggerman et al., 1993, Nature 365:445448; Ludwig et al., 1994, EMBO J. 13:44514458). Eag family members share about 47% amino acid sequence identity in their central hydrophobic cores and contain a segment homologous to a cyclic nucleotide-binding domain. The central hydrophobic cores of eag family members contain the six hydrophobic domains corresponding to the putative transmembrane domains (S1-S6) as well as a typical pore (P) region found in many other families of voltage-gated ion channels (Drysdale et al, 1991, Genetics 127:497-505; Warmke et al., 1991, Science 252:1560-1562; Guy et al., 1991, Science 254:730).

It soon became apparent that the eag family contained at least two other related subfamilies of genes, the elk (eag-like K⁺ channel) and erg (eag-related gene) subfamilies (Warmke & Ganetzky, 1994, Proc. Natl. Acad. Sci. USA 91:3438). Although the eag and elk members of this family are functionally typical delayed rectifier channels, the first member of the erg subfamily discovered, erg1, was found to have somewhat different biophysical properties. For example, although erg1 is, in fact, a delayed rectifier that conducts outward currents at voltages more positive than its activation threshold, its currents are also inwardly rectified to some degree (i.e., as the voltage is increased, the ability of the channel to conduct the outward currents is diminished) (Trudeau et al., 1995, Science 269:92-95). Human erg1 (h-erg1) is the locus for the LQT2 form of long-QT syndrome, an inherited disorder characterized by a prolonged QT interval in the electrocardiogram, placing those affected at risk of sudden death due to cardiac ventricular arrhythmia (Curran et al., 1995, Cell 80:795).

Other members of the erg subfamily have been identified. Shi et al., 1997, J. Neurosci. 17:9423-9432 identified two rat genes, erg2 and erg3, expressed exclusively in the rat nervous system that are highly similar to erg1.

It is desirable to discover as wide a variety as possible of novel potassium channel subunits, especially those from humans and those exhibiting restricted tissue expression. Such novel subunits would be attractive targets for drug discovery, useful in counterscreens for a variety of other drug targets, and would be valuable research tools for understanding more about ion channel biology.

### SUMMARY OF THE INVENTION

The present invention is directed to a novel human DNA sequence encoding human erg2 (h-erg2), a potassium channel subunit. The present invention includes certain splice variants and polymorphic variants of h-erg2. The present invention includes DNA comprising the nucleotide sequences shown as SEQ. ID. NOs.:1, 3, 5, and 7 as well as DNA comprising the coding regions of SEQ. ID. NOs.: 1, 3, 5, and 7. Also provided are the deduced protein sequences encoded by the novel DNA sequences. The human h-erg2 proteins of the present invention comprise the amino acid sequences shown as SEQ. ID. NOs.:2, 4, 6, and 8 as well as fragments thereof. Methods of expressing the novel h-erg2 potassium channel subunit proteins in recombinant systems are provided. Also provided are methods of using h-erg2 as a drug target by identifying activators and inhibitors of potassium channels comprising the h-erg2 subunits. Also provided are methods of using the novel h-erg2 subunits and DNA encoding these subunits in counterscreens for assays designed to identify activators and inhibitors of other drug targets.

### BRIEF DESCRIPTION OF THE DRAWINGS

FIG. 1A shows a cDNA sequence encoding h-erg2 (SEQ. ID. NO.:1) and FIG. 1B shows the corresponding amino acid sequence (SEQ. ID. NO.:2). The start ATG codon in FIG. 1A is at position 57-59; the stop codon is at position 2931-2933.

FIG. 2A shows a cDNA sequence encoding h-erg2 with a single nucleotide polymorphism (SEQ. ID. NO.:3) as compared to SEQ. ID. NO.:1. Position 2722 in SEQ. ID. NO.:3 is C rather than T as in SEQ. ID. NO.:1. FIG. 2B shows the amino acid sequence (SEQ. ID. NO.:4) encoded by SEQ. ID. NO.:3. SEQ. ID. NO.:4 differs from SEQ. ID. NO.:2 in having a T rather than an M at position 889.

FIG. 3A shows a cDNA sequence encoding a putative splice variant of h-erg2 with a 108 base pair insert at position 2289 (SEQ. ID. NO.:5) as compared to SEQ. ID. NO.:1. The insert is underlined. FIG. 3B shows the amino acid sequence (SEQ. ID. NO.:6) encoded by SEQ. ID. NO.:5.

FIG. 4A shows a cDNA sequence encoding a putative splice variant of h-erg2 with a 237 base pair deletion beginning at position 2637 (SEQ. ID. NO.:7) as compared to SEQ. ID. NO.:1. FIG. 4B shows the amino acid sequence (SEQ. ID. NO.:8) encoded by SEQ. ID. NO.:7.

FIGS. 5A-B shows an amino acid sequence alignment of h-erg2 (SEQ. ID. NO.:2), human erg1 (h-erg1) (SEQ. ID. NO.:9; GenBank accession no. U04270), and rat erg2 (SEQ. ID. NO.:10; GenBank accession no. AF016192). The consensus sequence is SEQ. ID. NO.:23.

FIG. 6 shows the results of a multi-tissue Northern blot demonstrating that h-erg2 is expressed specifically in kidney and prostate. The lanes were as follows: 1=heart; 2=brain; 3=placenta; 4=lung; 5=liver; 6=skeletal muscle; 7=kidney; 8=pancreas; 9=spleen; 10=thymus; 11=prostate; 12=testis; 13=uterus; 14=small intestine; 15=colon; 16=peripheral blood leukocytes.

### DETAILED DESCRIPTION OF THE INVENTION

For the purposes of this invention:

“Substantially free from other proteins” means at least 90%, preferably 95%, more preferably 99%, and even more preferably 99.9%, free of other proteins. Thus, a h-erg2 protein preparation that is substantially free from other proteins will contain, as a percent of its total protein, no more than 10%, preferably no more than 5%, more preferably no more than 1%, and even more preferably no more than 0.1%, of proteins that are not h-erg2 proteins. Whether a given h-erg2 protein preparation is substantially free from other proteins can be determined by conventional techniques of assessing protein purity such as, e.g., sodium dodecyl sulfate polyacrylamide gel electrophoresis (SDS-PAGE) combined with appropriate detection methods, e.g., silver staining or immunoblotting.

“Substantially free from other nucleic acids” means at least 90%, preferably 95%, more preferably 99%, and even more preferably 99.9%, free of other nucleic acids. Thus, a h-erg2 DNA preparation that is substantially free from other nucleic acids will contain, as a percent of its total nucleic acid, no more than. 10%, preferably no more than 5%, more preferably no more than 1%, and even more preferably no more than 0.1%, of nucleic acids that are not h-erg2 nucleic acids. Whether a given h-erg2 DNA preparation is substantially free from other nucleic acids can be determined by conventional techniques of assessing nucleic acid purity such as, e.g., agarose gel electrophoresis combined with appropriate staining methods, e.g., ethidium bromide staining.

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid); substitution of one aromatic amino acid (tryptophan, tyrosine, or phenylalanine) for another.

A polypeptide has “substantially the same biological activity as h-erg2” if that polypeptide is able to either form a functional potassium channel by itself, i.e., as a homomultimer, having properties similar to that of h-erg2 channels, or combine with at least one other potassium channel subunit so as to form a complex that constitutes a functional potassium channel where the polypeptide confers upon the complex (as compared with the other subunit alone) altered electrophysiological or pharmacological properties that are similar to the electrophysiological or pharmacological properties that the h-erg2 protein having SEQ. ID. NO.:2 confers on the other subunit and where the polypeptide has an amino acid sequence that is at least about 50% identical, preferably at least about 80% identical, and even more preferably at least about 95% identical to SEQ. ID. NO.:2 when measured by such standard programs as BLAST or FASTA. See, e.g., Gish & States, 1993, Nature Genetics 3:266-272 and Altschul et al., 1990, J. Mol. Biol. 215:403-410 for examples of sequence comparison programs.

The present invention relates to the identification and cloning of DNA encoding the human erg2 (h-erg2) protein. Although cDNA encoding rat erg2 has been isolated (Shi et al., 1997, J. Neurosci. 17:9423-9432 and GenBank accession no. AF016192), DNA encoding the complete, correct human erg2 (h-erg2) has not previously been reported. An EST that may represent a portion of h-erg2 was deposited in GenBank at accession no. H96170. Another sequence, X56415 (geneseqn database) may be a partial sequence of a h-erg2-related gene.

The present invention provides cDNA encoding h-erg2 having SEQ. ID. NO.:1. SEQ. ID. NO.:1 encodes a h-erg2 protein having SEQ. ID. NO.:2. Other sequence variants of h-erg2 have also been identified:

1. A single nucleotide polymorphism (T or C) was identified at nucleotide 2722 in SEQ. ID. NO.:1. The cDNA sequence corresponding to this polymorphism is SEQ. ID. NO.:3, which encodes a protein having SEQ. ID. NO.:4. Two clones were identified with a T and two clones with a C at position 2722, resulting in either a methionine (SEQ. ID. NO.:2) or threonine (SEQ. ID. NO.:4) at amino acid position 889 of the deduced amino acid sequence.

Relevant cDNA Sequence:

| | | |
|---|---|---|
| 2701 | TGCCTCACCT GGCTGTGGCA A(T/C)GGACAAAA CTCTGGCACC ATCCTCAGAA | (SEQ.ID.NO.:11) |

Relevant Deduced Amino Acid Sequence:

851 RLPQGFLPPA QTPSYGDLDD CSPKHRNSSP RMPHLAVA(M/T)D KTLAPSSEQE (SEQ. ID. NO.:12)

2. A variant with an insertion, presumably a splice variant, was also identified (SEQ. ID. NO.:5). As compared with SEQ. ID. NO.:1, SEQ. ID. NO.:5 contains the 108 base pair insert shown below at position 2289.

The DNA sequence of the cDNA insertion is:

| | |
|---|---|
| 2289 | GGGTGTCCCC ATGAGCTGGG GCCCCAGTTC CCCTCTAAAG GCTACAGCCT |
| 2339 | CCTGGGTCCT GGGAGCCAGA ACTCCATGGG GGCAGGACCT TGTGCTCCAG |
| 2389 | GGCACCCA (a portion of SEQ.ID.NO.:5) |

The insertion of this sequence results in the production of a protein (SEQ. ID. NO.:6) having the following 36 amino acid insertion at position 745 as compared with SEQ. ID. NO.:2.

| | |
|---|---|
| 745 | GSPHELGPQF PSKGYSLLGP GSQNSMGAGP CAPGHP |
| | (a portion of SEQ.ID.NO.:6) |

3. Another variant, containing a deletion as compared with SEQ. ID. NO.:1 (also presumably a splice variant) was identified. This variant contained a 237 base pair deletion starting at nucleotide position 2637 of SEQ. ID. NO.:1. The DNA sequence of this variant is shown in SEQ. ID. NO.:7; the amino acid sequence of this variant is shown in SEQ. ID. NO.:8.

The deleted sequence of SEQ. ID. NO.:1 is shown below in bold and underlined.

| | |
|---|---|
| 2601 | GGGCCCAGGC TGCCCCAGGG CTTTCTGCCT CCTGCACAGA CCCCAAGCTA |
| 2651 | TGGGGACTTG GATGACTGTA GTCCAAAGCA CAGGAACTCC TCCCCCAGGA |
| 2701 | TGCCTCACCT GGCTGTGGCA ATGGACAAAA CTCTGGCACC ATCCTCAGAA |
| 2751 | CAGGAACAGC CTGAGGGGCT CTGGCCACCC CTAGCCTCAC CTCTACATCC |
| 2801 | CCTGGAAGTA CAAGGACTCA TCTGTGGTCC CTGCTTCTCC TCCCTCCCTG |
| 2851 | AACACCTTGG CTCTGTTCCC AAGCAGCTGG ACTTCCAGAG ACATGGCTCA |
| | (a portion of SEQ.ID.NO.:1) |

This results in a deletion of 79 amino acids of the deduced amino acid sequence, starting at amino acid 861 of SEQ. ID. NO.:2.

The deleted amino acid sequence of SEQ. ID. NO.:2 is shown below in bold and underlined.

851 RLPQGFLPPA OTPSYGDLDD CSPKHRNSSP RMPHLAVAMD KTLAPSSEQE901 OPEGLWPPLA SPLHPLEVOG LICGPCFSSL PEHLGSVPKQ LDFQRHGSDP (a portion of SEQ. ID. NO.:2)

Northern blot analyses demonstrated strong expression of h-erg2 in kidney and prostate. All of the h-erg2 variants disclosed herein were cloned from kidney. This pattern of expression suggests that the h-erg2 potassium channel subunit may have therapeutic relevance for the treatment of hypo- and hypertension, renal failure, benign prostatic hyperplasia, prostate cancer, and infertility.

The present invention provides nucleic acids encoding the h-erg2 subunit that are substantially free from other nucleic acids. The nucleic acids may be DNA or RNA. The present invention also provides isolated and/or recombinant DNA molecules encoding the h-erg2 subunit. The present invention provides DNA molecules substantially free from other nucleic acids as well as isolated and/or recombinant DNA molecules comprising the nucleotide sequence shown in SEQ. ID. NOs.:1, 3, 5, or 7.

The present invention includes isolated DNA molecules as well as DNA molecules that are substantially free from other nucleic acids comprising the coding region of SEQ. ID. NOs.: 1, 3, 5, or 7. Accordingly, the present invention includes isolated DNA molecules and DNA molecules substantially free from other nucleic acids having a sequence comprising positions 57 to 2930 of SEQ. ID. NO.:1, 57 to 2930 of SEQ. ID. NO.:3, 57 to 3038 of SEQ. ID. NO.:5, or 57 to 2693 of SEQ. ID. NO.:7.

Also included are recombinant DNA molecules having a nucleotide sequence comprising positions 57 to 2930 of SEQ. ID. NO.:1, 57 to 2930 of SEQ. ID. NO.:3, 57 to 3038 of SEQ. ID. NO.:5, or 57 to 2693 of SEQ. ID. NO.:7. The novel DNA sequences of the present invention encoding the h-erg2 protein, in whole or in part, can be linked with other DNA sequences, i.e., DNA sequences to which DNA encoding the h-erg2 protein is not naturally linked, to form “recombinant DNA molecules” encoding the h-erg2 protein. Such other sequences can include DNA sequences that control transcription or translation such as, e.g., translation initiation sequences, internal ribosome entry sites, promoters for RNA polymerase II, transcription or translation termination sequences, enhancer sequences, sequences that control replication in microorganisms, sequences that confer antibiotic resistance, or sequences that encode a polypeptide “tag” such as, e.g., a polyhistidine tract, the FLAG epitope, or the myc epitope. The novel DNA sequences of the present invention can be inserted into vectors such as plasmids, cosmids, viral vectors, P1 artificial chromosomes, or yeast artificial chromosomes.

Included in the present invention are DNA sequences that hybridize to SEQ. ID. NO:1 under conditions of high stringency. By way of example, and not limitation, a procedure using conditions of high stringency is as follows: Prehybridization of filters containing DNA is carried out for 2 hr. to overnight at 65° C. in buffer composed of 6×SSC, 5× Denhardt's solution, and 100 μg/ml denatured salmon sperm DNA. Filters are hybridized for 12 to 48 hrs at 65° C. in prehybridization mixture containing 100 μg/ml denatured salmon sperm DNA and 5-20×10⁶ cpm of ³²P-labeled probe. Washing of filters is done at 37° C. for 1 hr in a solution containing 2×SSC, 0.1% SDS. This is followed by a wash in 0.1×SSC, 0.1% SDS at 50° C. for 45 min. before autoradiography.

Other procedures using conditions of high stringency would include either a hybridization carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Reagents mentioned in the foregoing procedures for carrying out high stringency hybridization are well known in the art. Details of the composition of these reagents can be found in, e.g., Sambrook, Fritsch, and Maniatis, 1989, Molecular Cloning: A Laboratory Manual, second edition, Cold Spring Harbor Laboratory Press. In addition to the foregoing, other conditions of high stringency which may be used are well known in the art.

The degeneracy of the genetic code is such that, for all but two amino acids, more than a single codon encodes a particular amino acid. This allows for the construction of synthetic DNA that encodes the h-erg2 protein where the nucleotide sequence of the synthetic DNA differs significantly from the nucleotide sequences of SEQ. ID. NO.:1, SEQ. ID. NO.:3, SEQ. ID. NO.:5, or SEQ. ID. NO.:7 but still encodes the same h-erg2 protein as SEQ. ID. NO.:1, SEQ. ID. NO.:3, SEQ. ID. NO.:5, or SEQ. ID. NO.:7. Such synthetic DNAs are intended to be within the scope of the present invention.

Mutated forms of SEQ. ID. NO.:1, SEQ. ID. NO.:3, SEQ. ID. NO.:5, or SEQ. ID. NO.:7 are intended to be within the scope of the present invention. In particular, mutated forms of SEQ. ID. NO.:1, SEQ. ID. NO.:3, SEQ. ID. NO.:5, or SEQ. ID. NO.:7 encoding a protein that forms potassium channels having altered voltage sensitivity, current carrying properties, or other properties as compared to potassium channels formed by the proteins encoded by SEQ. ID. NO.:1, SEQ. ID. NO.:3, SEQ. ID. NO.:5, or SEQ. ID. NO.:7, are within the scope of the present invention. Such mutant forms can differ from SEQ. ID. NO.:1, SEQ. ID. NO.:3, SEQ. ID. NO.:5, or SEQ. ID. NO.:7 by having nucleotide deletions, substitutions, or additions.

Also intended to be within the scope of the present invention are RNA molecules having sequences corresponding to SEQ. ID. NO.:1, SEQ. ID. NO.:3, SEQ. ID. NO.:5, or SEQ. ID. NO.:7 or corresponding to the coding regions of SEQ. ID. NO.:1, SEQ. ID. NO.:3, SEQ. ID. NO.:5, or SEQ. ID. NO.:7. The RNA molecules can be substantially free from other nucleic acids or can be isolated and/or recombinant RNA molecules. Antisense nucleotides, DNA or RNA, that are the reverse complements of SEQ. ID. NO.:1, SEQ. ID. NO.:3, SEQ. ID. NO.:5, or SEQ. ID. NO.:7, or portions thereof, are also within the scope of the present invention. In addition, polynucleotides based on SEQ. ID. NO.:1, SEQ. ID. NO.:3, SEQ. ID. NO.:5, or SEQ. ID. NO.:7 in which a small number of positions are substituted with non-natural or modified nucleotides such as inosine, methyl-cytosine, or deaza-guanosine are intended to be within the scope of the present invention. Polynucleotides of the present invention can also include sequences based on SEQ. ID. NO.:1, SEQ. ID. NO.:3, SEQ. ID. NO.:5, or SEQ. ID. NO.:7 but in which non-natural linkages between the nucleotides are present. Such non-natural linkages can be, e.g., methylphosphonates, phosphorothioates, phosphorodithionates, phosphoroamidites, and phosphate esters. Polynucleotides of the present invention can also include sequences based on SEQ. ID. NO.:1, SEQ. ID. NO.:3, SEQ. ID. NO.:5, or SEQ. ID. NO.:7 but having de-phospho linkages as bridges between nucleotides, e.g., siloxane, carbonate, carboxymethyl ester, acetamidate, carbamate, and thioether bridges. Other internucleotide linkages that can be present include N-vinyl, methacryloxyethyl, methacrylamide, or ethyleneimine linkages. Peptide nucleic acids based upon SEQ. ID. NO.:1, SEQ. ID. NO.:3, SEQ. ID. NO.:5, or SEQ. ID. NO.:7 are also included in the present invention. Generally, such polynucleotides comprising non-natural or modified nucleotides and/or non-natural linkages between the nucleotides, as well as peptide nucleic acids, will encode the same, or highly similar, proteins as are encoded by SEQ. ID. NO.:1, SEQ. ID. NO.:3, SEQ. ID. NO.:5, or SEQ. ID. NO.:7.

Another aspect of the present invention includes host cells that have been engineered to contain and/or express DNA sequences encoding the h-erg2 protein. Such recombinant host cells can be cultured under suitable conditions to produce h-erg2 protein. An expression vector containing DNA encoding h-erg2 protein can be used for the expression of h-erg2 protein in a recombinant host cell. Recombinant host cells may be prokaryotic or eukaryotic, including but not limited to, bacteria such as E. coli, fungal cells such as yeast, mammalian cells including, but not limited to, cell lines of human, bovine, porcine, monkey and rodent origin, amphibian cells such as Xenopus oocytes, and insect cells including but not limited to Drosophila and silkworm derived cell lines (e.g., Spodoptera frugiperda). Cells and cell lines which are suitable for recombinant expression of h-erg2 protein and which are widely available, include but are not limited to, L cells L-M(TK-) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), MRC-5 (ATCC CCL 171), CPAE (ATCC CCL 209), Saos-2 (ATCC HTB-85), ARPE-19 human retinal pigment epithelium (ATCC CRL-2302), Xenopus melanophores, and Xenopus oocytes.

A variety of mammalian expression vectors can be used to express recombinant h-erg2 protein in mammalian cells. Commercially available mammalian expression vectors which are suitable include, but are not limited to, pMC1neo (Stratagene), pSG5 (Stratagene), pcDNAI and pcDNAIamp, pcDNA3, pcDNA3.1, pCR3.1 (Invitrogen), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pIZD35 (ATCC 37565), and pSV2-dhfr (ATCC 37146). Another suitable vector is the PT7TS oocyte expression vector.

Following expression in recombinant cells, h-erg2 protein can be purified by conventional techniques to a level that is substantially free from other proteins. Techniques that can be used include ammonium sulfate precipitation, hydrophobic or hydrophilic interaction chromatography, ion exchange chromatography, affinity chromatography, phosphocellulose chromatography, size exclusion chromatography, preparative gel electrophoresis, and alcohol precipitation. In some cases, it may be advantageous to employ protein denaturing and/or refolding steps in addition to such techniques.

Certain potassium channel subunit proteins have been found to require the expression of other potassium channel subunits in order to be properly expressed at high levels and inserted in membranes. For example, some voltage-gated potassium channel Kvα subunits require other related α subunits or Kvβ subunits (Shi et al., 1995, Neuron 16:843-852) to form functional channels. As one specific example, co-expression of KCNQ3 appears to enhance the expression of KCNQ2 in Xenopus oocytes (Wang et al., 1998, Science 282:1890-1893). Accordingly, the recombinant expression of h-erg2 proteins may under certain circumstances benefit from the co-expression of other potassium channel proteins and such co-expression is intended to be within the scope of the present invention. Such co-expression can be effected by transfecting an expression vector encoding h-erg2 protein into a cell that naturally expresses another potassium channel protein. Alternatively, an expression vector encoding h-erg2 protein can be transfected into a cell in which an expression vector encoding another potassium channel protein has also been transfected. Preferably, such a cell does not naturally express h-erg2 subunit protein or the other potassium channel proteins.

The present invention includes h-erg2 proteins substantially free from other proteins. The deduced amino acid sequences of full-length h-erg2 subunit proteins are shown in SEQ. ID. NOs.:2, 4, 6, and 8. Thus, the present invention includes h-erg2 protein substantially free from other proteins comprising an amino acid sequence selected from the group consisting of SEQ. ID. NOs.:2, 4, 6, and 8. The present invention also includes isolated h-erg2 protein comprising an amino acid sequence selected from the group consisting of SEQ. ID. NOs.:2, 4, 6, and 8.

Mutated forms of h-erg2 proteins are intended to be within the scope of the present invention. In particular, mutated forms of SEQ. ID. NOs.:2, 4, 6, or 8 that form potassium channels having altered electrophysiological or pharmacological properties as compared to potassium, channels formed by SEQ. ID. NOs.:2, 4, 6, or 8 are within the scope of the present invention.

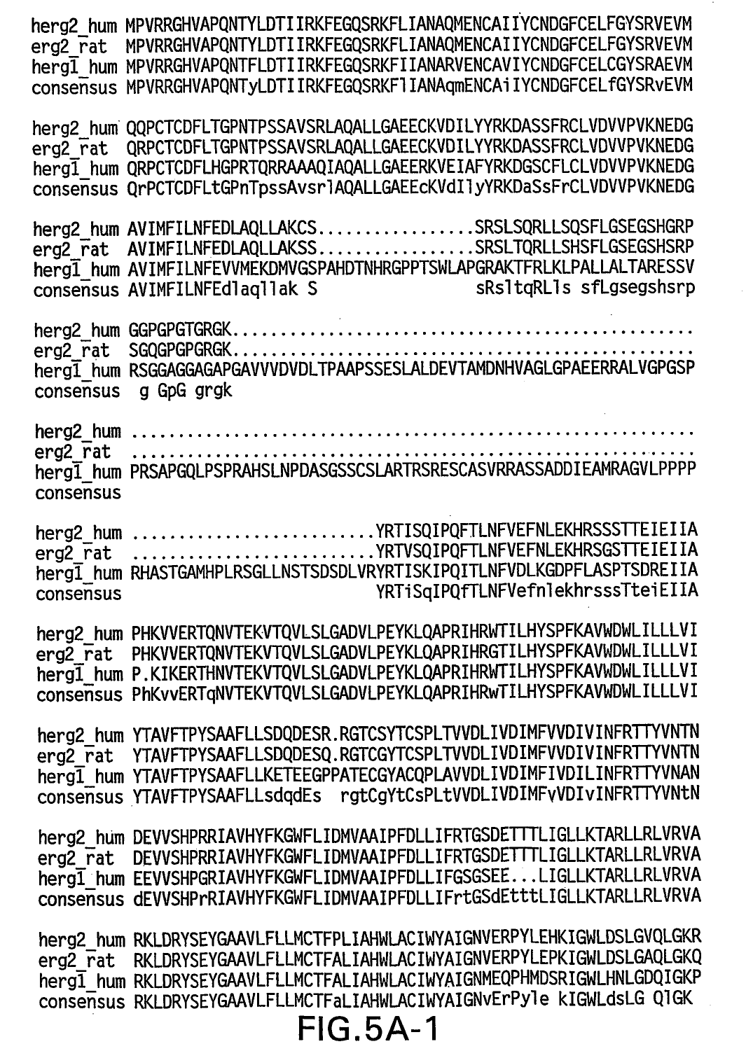

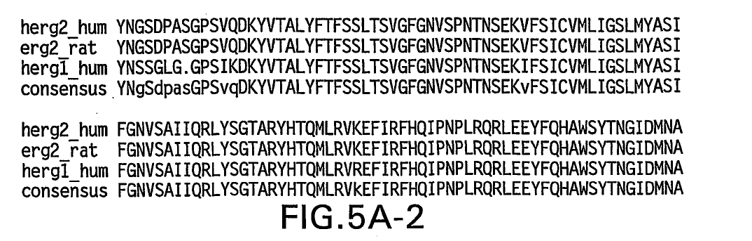

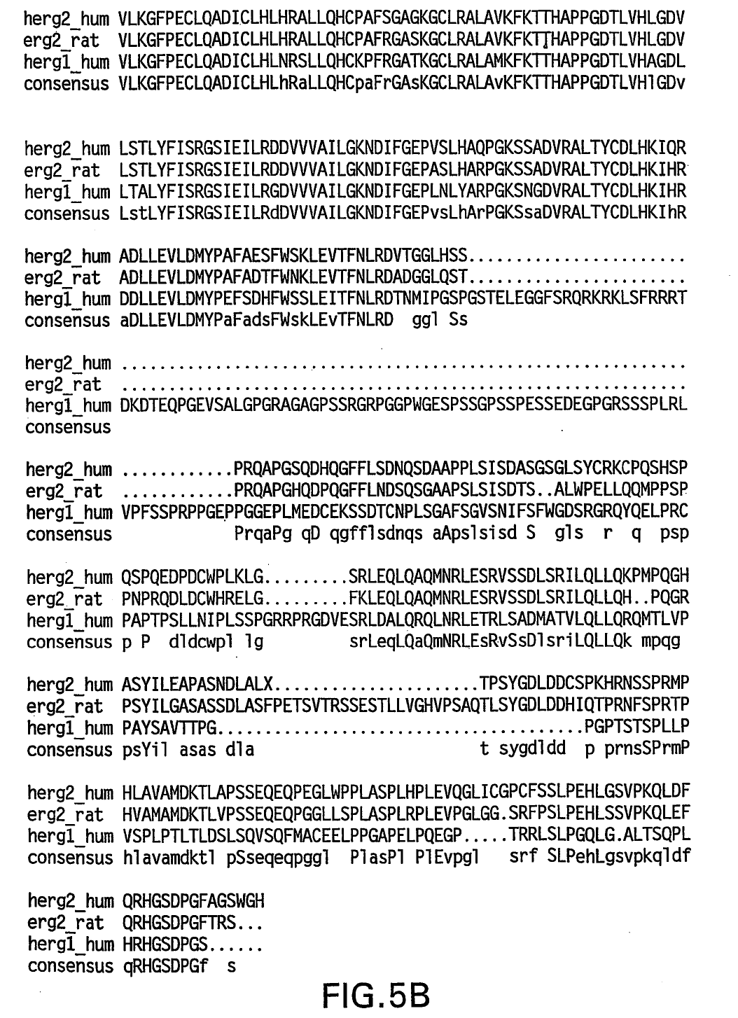

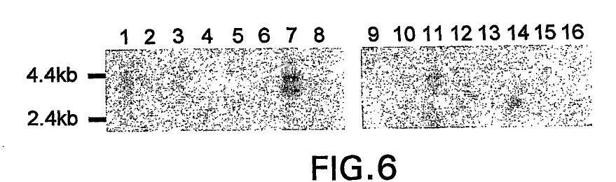

As with many proteins, it may be possible to modify many of the amino acids of the h-erg2 protein and still retain substantially the same biological activity as for the original protein. Thus, the present invention includes modified h-erg2 proteins which have amino acid deletions, additions, or substitutions but that still retain substantially the same biological activity as naturally occurring h-erg2 proteins. It is generally accepted that single amino acid substitutions do not usually alter the biological activity of a protein (see, e.g., Molecular Biology of the Gene, Watson et al., 1987, Fourth Ed., The Benjamin/Cummings Publishing Co., Inc., page 226; and Cunningham & Wells, 1989, Science 244:1081-1085). Accordingly, the present invention includes polypeptides where one amino acid substitution has been made in SEQ. ID. NOs.:2, 4, 6, or 8 wherein the polypeptides still retain substantially the same biological activity as naturally occurring h-erg2 proteins. The present invention also includes polypeptides where two or more amino acid substitutions have been made in SEQ. ID. NOs.:2, 4, 6, or 8 wherein the polypeptides still retain substantially the same biological activity as naturally occurring h-erg2 proteins. In particular, the present invention includes embodiments where the above-described substitutions are conservative substitutions. In particular, the present invention includes embodiments where the above-described substitutions do not occur in conserved positions. Conserved positions are those positions in which the h-erg2 protein having SEQ. ID. NO.:2, the herg1 protein (SEQ. ID. NO.:9), and the rat erg2 protein (SEQ. ID. NO.:10) share the same amino acid (see FIG. 5).

The h-erg2 proteins of the present invention may contain post-translational modifications, e.g., covalently linked carbohydrate, phosphorylation, myristoylation, palmytoylation, etc.

The present invention also includes chimeric h-erg2 proteins. Chimeric h-erg2 proteins consist of a contiguous polypeptide sequence of at least a portion of a h-erg2 protein fused to a polypeptide sequence that is not from a h-erg2 protein. The portion of the h-erg2 protein must include at least 10, preferably at least 25, and most preferably at least 50 contiguous amino acids from SEQ. ID. NO.:2, 4, 6, or 8.

The present invention also includes isolated h-erg2 protein and isolated DNA encoding h-erg2 protein. Use of the term “isolated” indicates that the h-erg2 protein or DNA has been removed from its normal cellular environment. Thus, an isolated h-erg2 protein may be in a cell-free solution or placed in a different cellular environment from that in which it occurs naturally. The term isolated does not necessarily imply that an isolated h-erg2 protein is the only, or predominant, protein present (although that is one of the meanings of isolated), but instead means that the isolated h-erg2 protein is at least 95% free of non-amino acid material (e.g., nucleic acids, lipids, carbohydrates) naturally associated with the h-erg2 protein.

It is known that certain potassium channel subunits can interact to form heteromeric complexes resulting in functional potassium channels. For example, KCNQ2 and KCNQ3 can assemble to form a heteromeric functional potassium channel (Wang et al., 1998, Science 282:1890-1893). Accordingly, it is believed that the h-erg2 proteins of the present invention may also be able to form heteromeric structures with other proteins where such heteromeric structures form functional potassium channels. Thus, the present invention includes such heteromers comprising h-erg2 protein.

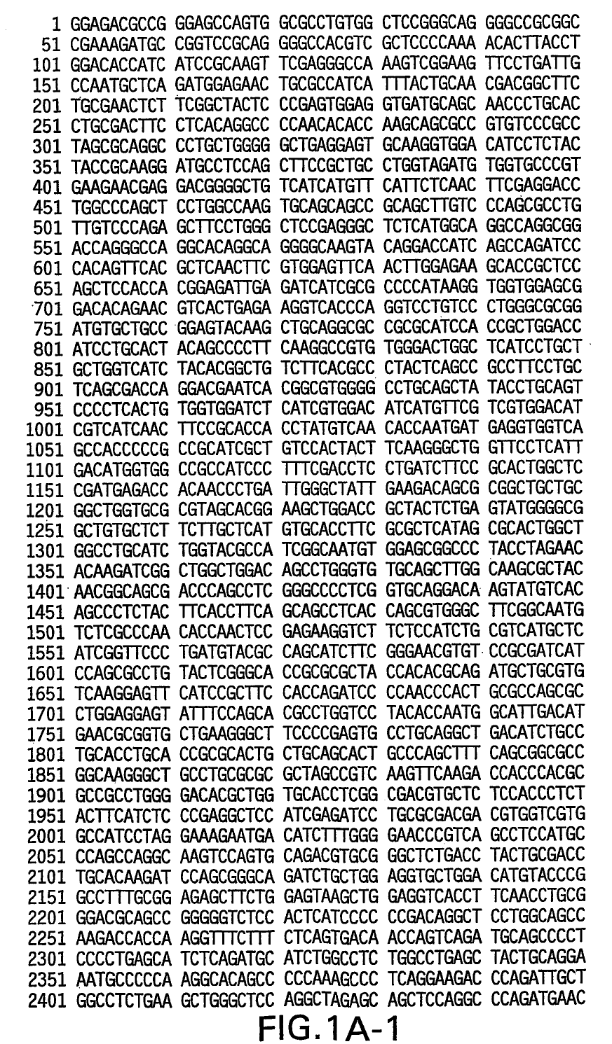

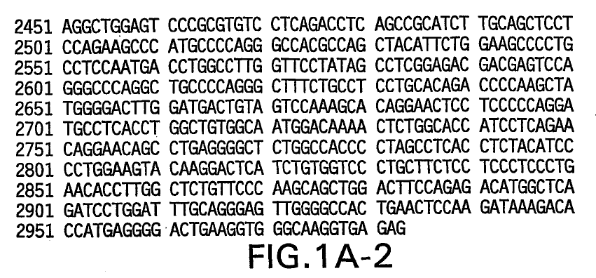

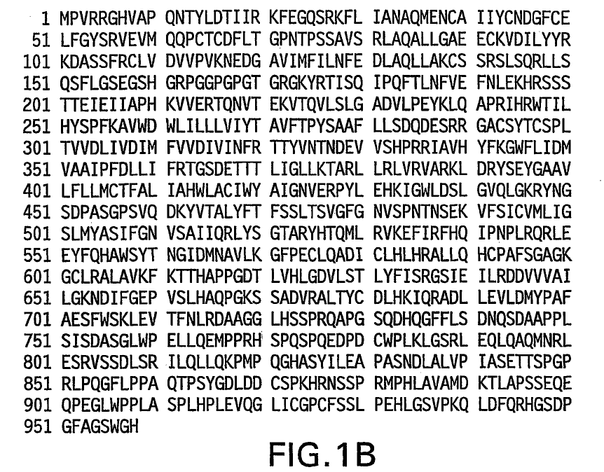

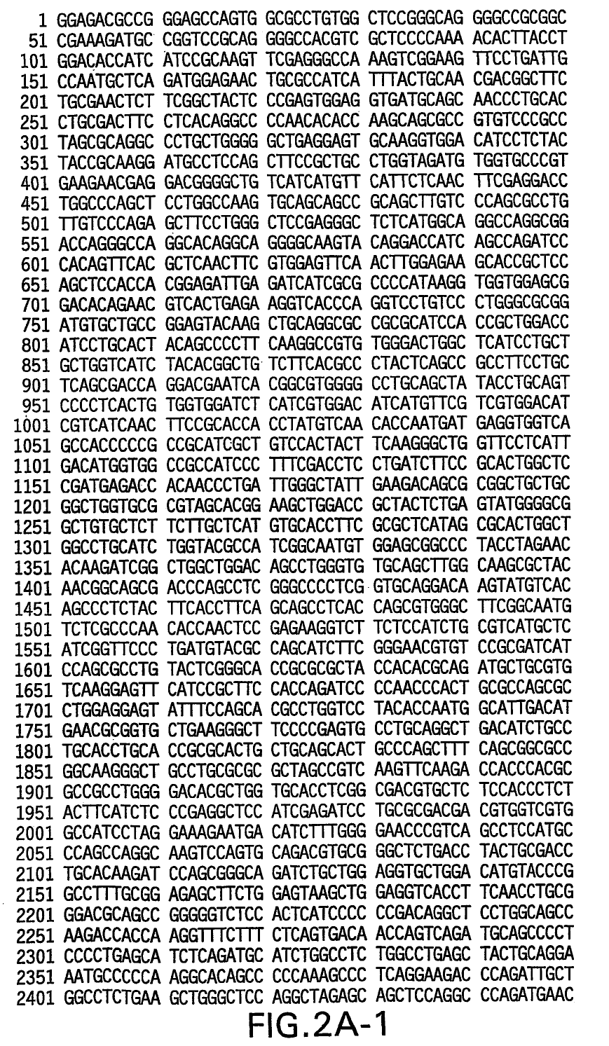

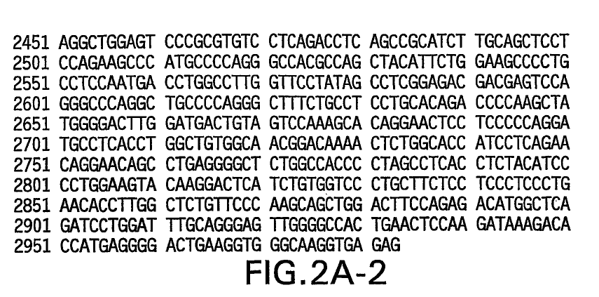

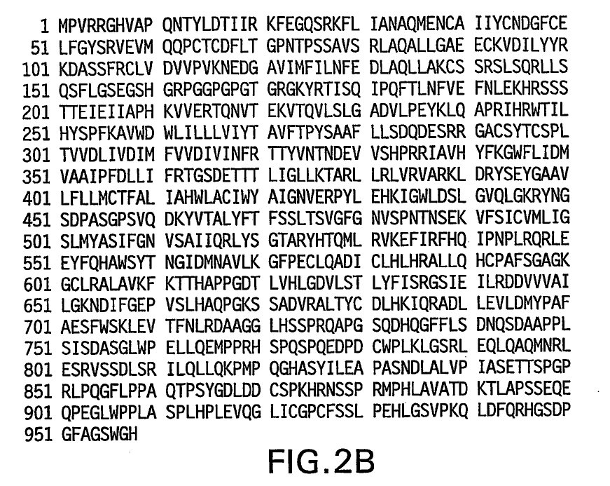

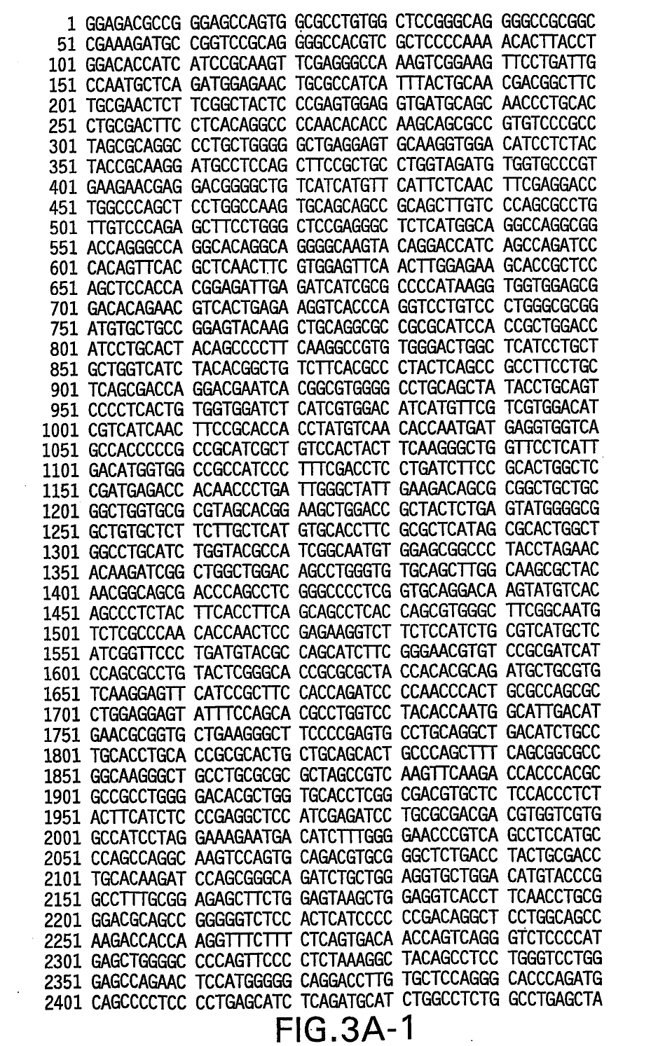

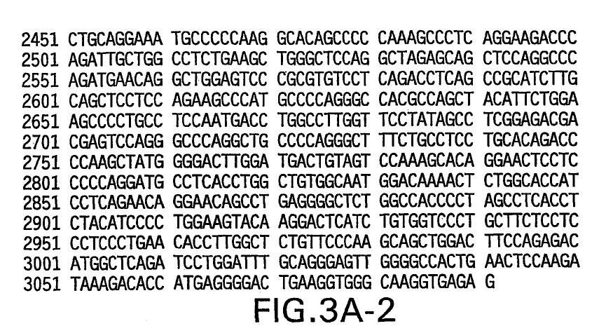

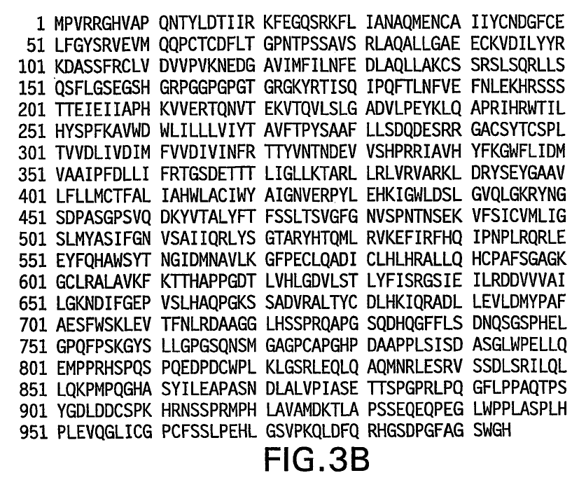

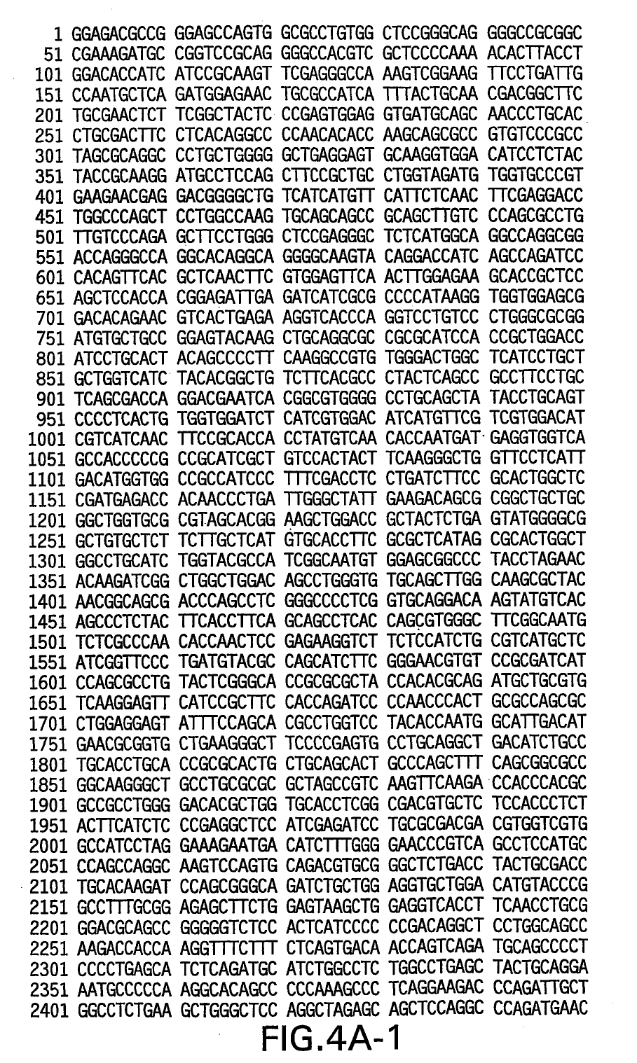

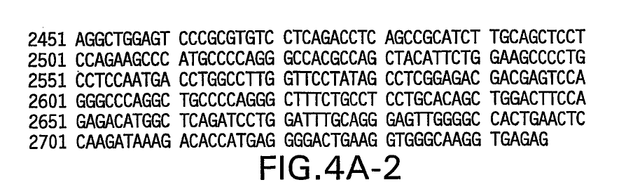

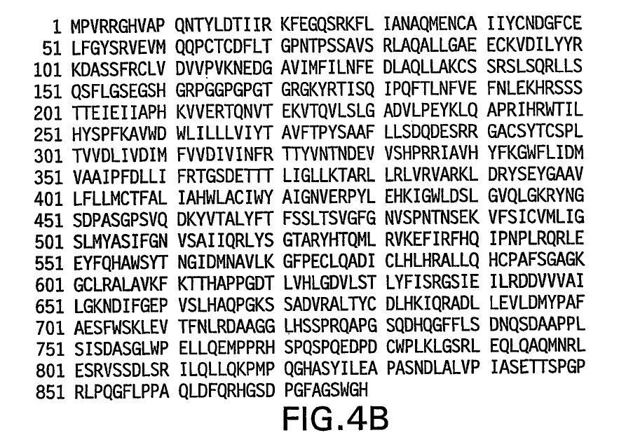

DNA encoding h-erg2 proteins can be obtained by methods well known in the art. For example, a cDNA fragment encoding full-length h-erg2 protein can be isolated from human kidney or prostate cDNA by using the polymerase chain reaction (PCR) employing suitable primer pairs. Such primer pairs can be selected based upon the DNA sequences encoding the h-erg2 proteins shown in FIGS. 1-4 as SEQ. ID. NOs.:1, 3, 5, and 7. Suitable primer pairs would be, e.g.:

| | |
|---|---|
| 5′ GGAGACGCCG GGAGCCAGTG GCGCC 3′ | (SEQ.ID.NO.:13) |
| 5′ CTC TCACCTTGCC CACCTTCAGT CCCC 3′ | (SEQ.ID.NO.:14) |

The above primers are meant to be illustrative only; one skilled in the art would readily be able to design other suitable primers based upon SEQ. ID. NOs.:1, 3, 5, and 7. Such primers could be produced by methods of oligonucleotide synthesis that are well known in the art.

PCR reactions can be carried out with a variety of thermostable enzymes including but not limited to AmpliTaq, AmpliTaq Gold, or Vent polymerase. For AmpliTaq, reactions can be carried out in 10 mM Tris-Cl, pH 8.3, 2.0 mM MgCl₂, 200 μM of each DNTP, 50 mM KCl, 0.2 μM of each primer, 10 ng of DNA template, 0.05 units/μl of AmpliTaq. The reactions are heated at 95° C. for 3 minutes and then cycled 35 times using the cycling parameters of 95° C., 20 seconds, 62° C., 20 seconds, 72° C., 3 minutes. In addition to these conditions, a variety of suitable PCR protocols can be found in PCR Primer, A Laboratory Manual, edited by C. W. Dieffenbach and G. S. Dveksler, 1995, Cold Spring Harbor Laboratory Press; or PCR Protocols: A Guide to Methods and Applications, Michael et al., eds., 1990, Academic Press.

Since the h-erg2 proteins of the present invention are highly homologous to other potassium channel subunit proteins, it is desirable to sequence the clones obtained by the herein-described methods, in order to verify that the desired h-erg2 protein has in fact been obtained. Sequencing is also advisable in order to ensure that one has obtained the desired cDNA from among SEQ. ID. NO.:1, 3, 5, and 7.

By these methods, cDNA clones encoding h-erg2 proteins can be obtained. These cDNA clones can be cloned into suitable cloning vectors or expression vectors, e.g., the mammalian expression vector pcDNA3.1 (Invitrogen, San Diego, Calif.). H-erg2 protein can then be produced by transferring expression vectors encoding h-erg2 or portions thereof into suitable host cells and growing the host cells under appropriate conditions. H-erg2 protein can then be isolated by methods well known in the art.

As an alternative to the above-described PCR methods, cDNA clones encoding h-erg2 proteins can be isolated from cDNA libraries using as a probe oligonucleotides specific for h-erg2 and methods well known in the art for screening cDNA libraries with oligonucleotide probes. Such methods are described in, e.g., Sambrook et al., 1989, Molecular Cloning: A Laboratory Manual; Cold Spring Harbor Laboratory, Cold Spring Harbor, N.Y.; Glover, D. M. (ed.), 1985, DNA Cloning: A Practical Approach, MRL Press, Ltd., Oxford, U.K., Vol. I, II. Oligonucleotides that are specific for h-erg2 and that can be used to screen cDNA libraries can be readily designed based upon the DNA sequences shown in FIGS. 1-4 (viz., SEQ. ID. NOs.:1, 3, 5, and 7) and can be synthesized by methods well-known in the art.

Genomic clones containing the h-erg2 gene can be obtained from commercially available human PAC or BAC libraries available from Research Genetics, Huntsville, Ala. Alternatively, one may prepare genomic libraries, e.g., in P1 artificial chromosome vectors, from which genomic clones containing the h-erg2 gene can be isolated, using probes based upon the h-erg2 DNA sequences disclosed herein. Methods of preparing such libraries are known in the art (see, e.g., Ioannou et al., 1994, Nature Genet. 6:84-89).

The novel DNA sequences of the present invention can be used in various diagnostic methods The present invention provides diagnostic methods for determining whether a patient carries a mutation in the h-erg2 gene. In broad terms, such methods comprise determining the DNA sequence of a region in or near the h-erg2 gene from the patient and comparing that sequence to the sequence from the corresponding region of the h-erg2 gene from a non-affected person, i.e., a person who does not have the condition which is being diagnosed, where a difference in sequence between the DNA sequence of the gene from the patient and the DNA sequence of the gene from the non-affected person indicates that the patient has a mutation in the h-erg2 gene.

The present invention also provides oligonucleotide probes, based upon SEQ. ID. NOs.:1, 3, 5, or 7 that can be used in diagnostic methods to identify patients having mutated forms of the h-erg2 gene, to determine the level of expression of RNA encoding h-erg2, or to isolate genes homologous to h-erg2 from other species. In particular, the present invention includes DNA oligonucleotides comprising at least about 10, 15, or 18 contiguous nucleotides of SEQ. ID. NOs.:1, 3, 5, or 7 where the oligonucleotide probe comprises no stretch of contiguous nucleotides longer than 5 from: SEQ. ID. NOs.:1, 3, 5, or 7 other than the said at least about 10, 15, or 18 contiguous nucleotides. The oligonucleotides can be substantially free from other nucleic acids. Also provided by the present invention are corresponding RNA oligonucleotides. The DNA or RNA oligonucleotides can be packaged in kits.

The present invention makes possible the recombinant expression of h-erg2 protein in various cell types. Such recombinant expression makes possible the study of this protein so that its biochemical activity and its possible role in various diseases such as renal failure, hypokalemia, hypertension, hypotension, benign prostatic hyperplasia, infertility, and prostate cancer can be elucidated.

The present invention also makes possible the development of assays which measure the biological activity of potassium channels containing h-erg2 protein. Assays using recombinantly expressed h-erg2 protein are especially of interest. Such assays can be used to screen libraries of compounds or other sources of compounds to identify compounds that are activators or inhibitors of the activity of potassium channels containing h-erg2 protein. Such identified compounds can serve as “leads” for the development of pharmaceuticals that can be used to treat patients having diseases in which it is beneficial to enhance or suppress potassium channel activity.

In versions of the above-described assays, potassium channels containing mutant h-erg2 proteins are used and inhibitors or activators of the activity of the mutant potassium channels are identified.

Preferred cell lines for recombinant expression of h-erg2 proteins are those which do not express endogenous potassium channels (e.g., CV-1, NIH-3T3, CHO-K1, COS-7). Such cell lines can be exposed to ⁸⁶Rb, an ion which can pass through potassium channels. The influx of ⁸⁶Rb into such cells can be assayed in the presence and absence of collections of substances (e.g., combinatorial libraries, natural products, analogues of lead compounds produced by medicinal chemistry), or members of such collections, and those substances that are able to alter ⁸⁶Rb influx thereby identified. Such substances are likely to be activators or inhibitors of potassium channels containing h-erg2 protein. Similarly, the cells can be preloaded with ⁸⁶Rb and efflux of the ion initiated by opening of the potassium channels, e.g., by depolarization of the cells with potassium. The efflux of ⁸⁶Rb from such cells can then be assayed in the presence and absence of collections of substances (e.g., combinatorial libraries, natural products, analogues of lead compounds produced by medicinal chemistry), or members of such collections, and those substances that are able to alter ⁸⁶Rb efflux thereby identified. Such substances are likely to be activators or inhibitors of potassium channels containing h-erg2 protein.

Activators and inhibitors of potassium channels containing h-erg2 proteins are likely to be substances that are capable of binding to potassium channels containing h-erg2 proteins. Thus, one type of assay determines whether one or more of a collection of substances is capable of such binding.

Accordingly, the present invention provides a method of identifying substances that bind to potassium channels containing h-erg2 protein comprising:

(a) providing cells expressing a potassium channel containing h-erg2 protein;

(b) exposing the cells to a substance that is not known to bind potassium channels containing h-erg2 protein;

(c) determining the amount of binding of the substance to the cells;

(d) comparing the amount of binding in step (c) to the amount of binding of the substance to control cells where the control cells are substantially identical to the cells of step (a) except that the control cells do not express h-erg2 protein;

where if the amount of binding in step (c) is greater than the amount of binding of the substance to control cells, then the substance binds to potassium channels containing h-erg2 protein.

An example of control cells that are substantially identical to the cells of step (a) would be a parent cell line where the parent cell line is transfected with an expression vector encoding h-erg2 protein in order to produce the cells expressing a potassium channel containing h-erg2 protein of step (a).

Another version of this assay makes use of compounds that are known to bind to potassium channels containing h-erg2 protein. Substances that are new binders are identified by virtue of their ability to augment or block the binding of these known compounds. This can be done if the known compound is used at a concentration that is far below saturation, in which case a substance that is a new binder is likely to be able to either augment or block the binding of the known compound. Substances that have this ability are likely themselves to be inhibitors or activators of potassium channels containing h-erg2 protein.

Accordingly, the present invention includes a method of identifying substances that bind potassium channels containing h-erg2 protein and thus are likely to be inhibitors or activators of potassium channels containing h-erg2 protein comprising:

(a) providing cells expressing potassium channels containing h-erg2 protein;

(b) exposing the cells to a compound that is known to bind to the potassium channels containing h-erg2 protein in the presence and in the absence of a substance not known to bind to potassium channels containing h-erg2 protein;

(c) determining the amount of binding of the compound to the cells in the presence and in the absence of the substance not known to bind to potassium channels containing h-erg2 protein;

where if the amount of binding of the compound in the presence of the substance differs from that in the absence of the substance, then the substance binds potassium channels containing h-erg2 protein and is likely to be an inhibitor or activator of potassium channels containing h-erg2 protein.

Generally, the known compound is labeled (e.g., radioactively, enzymatically, fluorescently) in order to facilitate measuring its binding to the potassium channels.

Once a substance has been identified by the above-described methods, it can be assayed in functional tests, such as those described herein, in order to determine whether it is an inhibitor or an activator.

In particular embodiments, the compound known to bind potassium channels containing h-erg2 protein is selected from the group consisting of: MK-499, dofetilide, astemizole, terfenadine, E-4031, and ErgTx (Gurrola et al., 1999, FASEB J. 13:953-962).

The present invention includes a method of identifying activators or inhibitors of potassium channels containing h-erg2 protein comprising:

(a) recombinantly expressing h-erg2 protein in a host cell so that the recombinantly expressed h-erg2 protein forms potassium channels either by itself or by forming heteromers with other potassium channel subunit proteins;

(b) measuring the biological activity of the potassium channels formed in step (a) in the presence and in the absence of a substance suspected of being an activator or an inhibitor of potassium channels containing h-erg2 protein;

where a change in the biological activity of the potassium channels formed in step (a) in the presence as compared to the absence of the substance indicates that the substance is an activator or an inhibitor of potassium channels containing h-erg2 protein.

In particular embodiments of the methods described herein, the biological activity is the production of a potassium current, the influx of ⁸⁶Rb, the efflux of ⁸⁶Rb, the influx or efflux of NH₃, or a change in membrane potential caused by opening or closing of the h-erg2 containing channel.

In particular embodiments, it may be advantageous to recombinantly express the other subunits of potassium channels. Alternatively, it may be advantageous to use host cells that endogenously express such other subunits.

In particular embodiments, a vector encoding h-erg2 protein is transferred into Xenopus oocytes in order to cause the expression of h-erg2 protein in the oocytes. Alternatively, RNA encoding h-erg2 protein can be prepared in vitro and injected into the oocytes, also resulting in the expression of h-erg2 protein in the oocytes. Following expression of the h-erg2 protein in the oocytes, and following the formation of potassium channels containing h-erg2, membrane currents are measured after the transmembrane voltage is changed in steps. A change in membrane current is observed when the potassium channels open or close, modulating potassium ion flow. Similar studies were reported for KCNQ2 and KCNQ3 potassium channels in Wang et al., 1998, Science 282:1890-1893 and for minK channels by Goldstein & Miller, 1991, Neuron 7:403-408. These references and references cited therein can be consulted for guidance as to how to carry out such studies. In such studies it may be advantageous to co-express other potassium channel subunit proteins in addition to h-erg2 in the oocytes. Similar studies can be carried out in a variety of different types of host cells.

Inhibitors or activators of potassium channels containing h-erg2 protein can be identified by exposing the oocytes to individual substances or collections of substances and determining whether the substances can block/diminish or enhance the membrane currents observed in the absence of the substance.

Accordingly, the present invention provides a method of identifying inhibitors or activators of potassium channels containing h-erg2 protein comprising:

(a) expressing h-erg2 protein in cells such that potassium channels containing h-erg2 protein are formed;

(b) changing the transmembrane potential of the cells in the presence and the absence of a substance suspected of being an inhibitor or an activator of potassium channels containing h-erg2 protein;

(c) measuring membrane potassium currents following step (b);

where if the potassium membrane currents measured in step (c) are greater in the absence rather than in the presence of the substance, then the substance is an inhibitor of potassium channels containing h-erg2 protein;

where if the potassium membrane currents measured in step (c) are greater in the presence rather than in the absence of the substance, then the substance is an activator of potassium channels containing h-erg2 protein.

The present invention also includes assays for the identification of activators and inhibitors of potassium channels containing h-erg2 protein that are based upon fluorescence resonance energy transfer (FRET) between a first and a second fluorescent dye where the first dye is bound to one side of the plasma membrane of a cell expressing potassium channels containing h-erg2 protein and the second dye is free to shuttle from one face of the membrane to the other face in response to changes in membrane potential. In certain embodiments, the first dye is impenetrable to the plasma membrane of the cells and is bound predominately to the extracellular surface of the plasma membrane. The second dye is trapped within the plasma membrane but is free to diffuse within the membrane. At normal (i.e., negative) resting potentials of the membrane, the second dye is bound predominately to the inner surface of the extracellular face of the plasma membrane, thus placing the second dye in close proximity to the first dye. This close proximity allows for the generation of a large amount of FRET between the two dyes. Following membrane. depolarization, the second dye moves from the extracellular face of the membrane to the intracellular face, thus increasing the distance between the dyes. This increased distance results in a decrease in FRET, with a corresponding increase in fluorescent emission derived from the first dye and a corresponding decrease in the fluorescent emission from the second dye. In this way, the amount of FRET between the two dyes can be used to measure the polarization state of the membrane. For a description of this technique, see Gonzalez & Tsien, 1997, Chemistry & Biology 4:269-277. See also González & Tsien, 1995, Biophys. J. 69:1272-1280 and U.S. Pat. No. 5,661,035.

In certain embodiments, the first dye is a fluorescent lectin or a fluorescent phospholipid that acts as the fluorescent donor. Examples of such a first dye are: a coumarin-labeled phosphatidylethanolamine (e.g., N-(6-chloro-7-hydroxy-2-oxo-2H—1-benzopyran-3-carboxamidoacetyl)-dimyristoylphosphatidyl-ethanolamine) or N-(7-nitrobenz-2-oxa-1,3-diazol-4-yl)-dipalmitoylphosphatidylethanolamine); a fluorescently-labeled lectin (e.g., fluorescein-labeled wheat germ agglutinin). In certain embodiments, the second dye is an oxonol that acts as the fluorescent acceptor. Examples of such a second dye are: bis(1,3-dialkyl-2-thiobarbiturate)trimethineoxonols (e.g., bis(1,3-dihexyl-2-thiobarbiturate)trimethineoxonol) or pentamethineoxonol analogues (e.g., bis(1,3-dihexyl-2-thiobarbiturate)pentamethineoxonol; or bis(1,3-dibutyl-2-thiobarbiturate)pentamethineoxonol). See Gonzalez & Tsien, 1997, Chemistry & Biology 4:269-277 for methods of synthesizing various dyes suitable for use in the present invention. In certain embodiments, the assay may comprise a natural carotenoid, e.g., astaxanthin, in order to reduce photodynamic damage due to singlet oxygen.

The above described assays can be utilized to discover activators and inhibitors of potassium channels containing h-erg2 protein. Such assays will generally utilize cells that express potassium channels containing h-erg2 protein, e.g., by transfection with expression vectors encoding h-erg2 protein and, optionally, other potassium channel subunits.

The cellular membrane potential is determined by the balance between inward (depolarizing) and outward (repolarizing) ionic fluxes through various ion pumps and channels. Potassium channels such as h-erg2 are typically highly selective for K⁺ and, therefore, exhibit reversal potentials close to the potassium equilibrium potential (E_{K}). Such potassium channels thus function to maintain the resting membrane potential of a cell near E_{K}. The presence of an inhibitor of a potassium channel containing h-erg2 will prevent, or diminish, the ability of this channel to maintain this polarized (i.e., negative) membrane potential and the cell will, therefore, depolarize. Thus, membrane potential will tend to become more positive in the presence of h-erg2 inhibitors. Changes in membrane potential that are caused by inhibitors of potassium channels containing h-erg2 protein can be monitored by the assays using FRET described above.

Accordingly, the present invention provides a method of identifying inhibitors of potassium channels containing h-erg2 protein comprising:

(a) providing cells comprising:

(1) an expression vector that directs the expression of h-erg2 protein in the cells so that potassium channels containing h-erg2 protein are formed in the cells;

(2) a first fluorescent dye, where the first dye is bound to one side of the plasma membrane of the cells; and

(3) a second fluorescent dye, where the second fluorescent dye is free to distribute from one face of the plasma membrane of the cells to the other face in response to changes in membrane potential;

(b) exposing the cells to a substance that is suspected of being an inhibitor of potassium channels containing h-erg2 protein;

(c) measuring the amount of fluorescence resonance energy transfer (FRET) in the cells in the presence and in the absence of the substance;

(d) comparing the amount of FRET exhibited by the cells in the presence and in the absence of the substance;

where if the amount of FRET exhibited by the cells in the presence of the substance is less than the amount of FRET exhibited by the cells in the absence of the substance then the substance is an inhibitor of potassium channels containing h-erg2 protein.

The resting membrane potential of a cell depends on the balance of inward and outward currents that are active in that cell. Thus, if a K⁺ current, e.g., that produced by h-erg2, is the only or predominant current, the resting membrane potential will be near E_{K}. However, if counteracting depolarizing currents are also found in the cell, the resting membrane potential will be more depolarized and the exact value will depend on the relative magnitudes of the depolarizing and hyperpolarizing currents. One can therefore construct a cell line that expresses a potassium channel (e.g., h-erg2) and exhibits a more positive resting membrane potential than E_{K} by co-expression of a depolarizing current in the same cell. Examples of such depolarizing currents could be those carried by Na⁺, Ca⁺⁺, or Cl⁻ channels, ionophores, or combinations thereof. In these cells, an activator of the potassium channel would increase the relative contribution of the outward potassium current compared to the inward depolarizing current and, in doing so, make the membrane potential more negative (i.e., drive it closer to E_{K}). Changes in membrane potential that are caused by activators of potassium channels containing h-erg2 protein in such cells can be monitored by the assays using FRET described above.

Accordingly, the present invention provides a method of identifying activators of potassium channels containing h-erg2 protein comprising:

(a) providing cells comprising:

(1) expression vectors that direct the expression of h-erg2 protein and a depolarizing channel in the cells so that potassium channels containing h-erg2 protein and a depolarizing channel are both expressed in the same cells;

(2) a first fluorescent dye, where the first dye is bound to one side of the plasma membrane of the cells; and

(3) a second fluorescent dye, where the second fluorescent dye is free to distribute from one face of the plasma membrane of the cells to the other face in response to changes in membrane potential;

(b) exposing the cells to a substance suspected of being an activator of potassium channels containing the h-erg2 protein;

(c) measuring the amount of fluorescence resonance energy transfer (FRET) in the cells in the presence and in the absence of the substance;

(d) comparing the amount of FRET exhibited by the cells in the presence and in the absence of the substance;

wherein if the amount of FRET exhibited by the cells in the presence of the substance is greater than the amount of FRET exhibited by the cells in the absence of the substance then the substance is an activator of potassium channels containing h-erg2 protein.

The substances identified by the above-described method may either be activators of potassium channels containing h-erg2 protein or the substances may be inhibitors of the depolarizing current or currents. These two possibilities can be distinguished by expressing the depolarizing channels alone, i.e., without the potassium channels containing h-erg2 protein, in another cell line. The substances can then be tested against cells containing the depolarizing currents alone and it can thereby be determined if the substances are able to inhibit the depolarizing currents. Alternatively, these substances can be directly tested on the potassium channel containing h-erg2 in voltage clamp experiments to determine if they are activators of that channel.

The above-described method can be used to identify inhibitors of potassium channels containing h-erg2 protein as well. This is because the presence of an inhibitor will block or diminish current movement through the h-erg2 channels, thus preventing or lessening the ability of the h-erg2 channels to counteract the effect of the depolarizing channels. Thus, the present invention includes a method of identifying substances that may be inhibitors of potassium channels containing h-erg2 protein comprising:

(a) providing cells comprising:

(1) expression vectors that direct the expression of h-erg2 protein and a depolarizing channel in the cells so that potassium channels containing h-erg2 protein and a depolarizing channel are both expressed in the same cells;

(2) a first fluorescent dye, where the first dye is bound to one side of the plasma membrane of the cells; and

(3) a second fluorescent dye, where the second fluorescent dye is free to distribute from one face of the plasma membrane of the cells to the other face in response to changes in membrane potential;

(b) exposing the cells to a substance that is suspected of being an inhibitor of potassium channels containing h-erg2 protein;

(c) measuring the amount of fluorescence resonance energy transfer FRET) in the cells in the presence and in the absence of the substance;

(d) comparing the amount of FRET exhibited by the cells in the presence and in the absence of the substance;

wherein if the amount of FRET exhibited by the cells in the presence of the substance is less than the amount of FRET exhibited by the cells in the absence of the substance then the substance is an inhibitor of potassium channels containing h-erg2 protein.

The substances identified by the above-described method may either be inhibitors of potassium channels containing h-erg2 protein or the substances may be activators of the depolarizing current or currents. These two possibilities can be distinguished by expressing the depolarizing channels alone, i.e., without the potassium channels containing h-erg2 protein, in another cell line. The substances can then be tested against cells containing the depolarizing currents alone and it can thereby be determined if the substances are able to activate the depolarizing currents. Alternatively, these substances can be directly tested on the potassium channel containing h-erg2 in voltage clamp experiments to determine if they are inhibitors of the h-erg2 channel.

In particular embodiments of the above-described methods, the depolarizing channel is a sodium, calcium, non-specific cation, or chloride channel or an ionophore.

In order to be sure that the effect of the substance in the above-described assays is arising through its action at potassium channels containing h-erg2 protein, control experiments can be run in which the cells are as above, except that they do not contain an expression vector that directs the expression of h-erg2 protein.

In particular embodiments of the above-described methods, the expression vector is transfected into the test cells.

In particular embodiments of the above-described methods, the h-erg2 protein has an amino acid sequence selected from the group consisting of SEQ. ID. NOs.:2, 4, 6, and 8. In particular embodiments of the above-described methods, the expression vector comprises positions 57 to 2930 of SEQ. ID. NO.:1, 57 to 2930 of SEQ. ID. NO.:3, 57 to 3038 of SEQ. ID. NO.:5, or 57 to 2693 of SEQ. ID. NO.:7.

In particular embodiments of the above-described methods, the first fluorescent dye is selected from the group consisting of: a fluorescent lectin; a fluorescent phospholipid; a coumarin-labeled phosphatidylethanolamine; N-(6-chloro-7-hydroxy-2-oxo-2H—1-benzopyran-3-carboxamidoacetyl)-dimyristoylphosphatidyl-ethanolamine); N-(7-nitrobenz-2-oxa-1,3-diazol-4-yl)-dipalmitoylphosphatidylethanolamine); and fluorescein-labeled wheat germ agglutinin.

In particular embodiments of the above-described methods, the second fluorescent dye is selected from the group consisting of: an oxonol that acts as the fluorescent acceptor; bis(1,3-dialkyl-2-thiobarbiturate)trimethineoxonols; bis(1,3-dihexyl-2-thiobarbiturate)trimethineoxonol; bis(1,3-dialkyl-2-thiobarbiturate) quatramethineoxonols; bis(1,3-dialkyl-2-thiobarbiturate)pentamethineoxonols; bis(1,3-dihexyl-2-thiobarbiturate)pentamethineoxonbl; bis(1,3-dibutyl-2-thiobarbiturate)pentamethineoxonol); and bis(1,3-dialkyl-2-thiobarbiturate)hexamethineoxonols.

In a particular embodiment of the above-described methods, the cells are eukaryotic cells. In another embodiment, the cells are mammalian cells. In other embodiments, the cells are L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K₁ (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), or MRC-5 (ATCC CCL 171).

In assays to identify activators or inhibitors of potassium channels containing h-erg2 protein, it may be advantageous to co-express another potassium channel subunit besides h-erg2. In particular, it may be advantageous to co-express a related potassium channel subunit, such as erg1 or erg3. Preferably, this is done by co-transfecting into the cells an expression vector encoding the other subunit.

While the above-described methods are explicitly directed to testing whether “a” substance is an activator or inhibitor of potassium channels containing h-erg2 protein, it will be clear to one skilled in the art that such methods can be adapted to test collections of substances, e.g., combinatorial libraries, natural products extracts, to determine whether any members of such collections are activators or inhibitors of potassium channels containing h-erg2 protein. Accordingly, the use of collections of substances, or individual members or subsets of such members of such collections, as the substance in the above-described methods is within the scope of the present invention. In particular, it is envisioned that libraries that have been designed to incorporate chemical structures that are known to be associated with potassium ion channel modulation, e.g., dihydrobenzopyran libraries for potassium channel activators (International Patent Publication WO 95/30642) or biphenyl-derivative libraries for potassium channel inhibitors (International Patent Publication WO 95/04277), will be of special interest.

The present invention includes pharmaceutical compositions comprising activators or inhibitors of potassium channels comprising h-erg2 protein that have been identified by the herein-described methods. The activators or inhibitors are generally combined with pharmaceutically acceptable carriers to form pharmaceutical compositions. Examples of such carriers and methods of formulation of pharmaceutical compositions containing activators or inhibitors and carriers can be found in Gennaro, ed., Remington's Pharmaceutical Sciences, 18^{th} Edition, 1990, Mack Publishing Co., Easton, Pa. To form a pharmaceutically acceptable composition suitable for effective administration, such compositions will contain a therapeutically effective amount of the activators or inhibitors.

Therapeutic or prophylactic compositions are administered to an individual in amounts sufficient to treat or prevent conditions where the activity of potassium channels containing h-erg2 protein is abnormal. The effective amount can vary according to a variety of factors such as the individual's condition, weight, gender, and age. Other factors include the mode of administration. The appropriate amount can be determined by a skilled physician. Generally, an effective amount will be from about 0.01 to about 1,000, preferably from about 0.1 to about 250, and even more preferably from about 1 to about 50 mg per adult human per day.

Compositions can be used alone at appropriate dosages. Alternatively, co-administration or sequential administration of other agents can be desirable.

The compositions can be administered in a wide variety of therapeutic dosage forms in conventional vehicles for administration. For example, the compositions can be administered in such oral dosage forms as tablets, capsules (each including timed release and sustained release formulations), pills, powders, granules, elixirs, tinctures, solutions, suspensions, syrups and emulsions, or by injection. Likewise, they can also be administered in intravenous (both bolus and infusion), intraperitoneal, subcutaneous, topical with or without occlusion, or intramuscular form, all using forms well known to those of ordinary skill in the pharmaceutical arts.

Compositions can be administered in a single daily dose, or the total daily dosage can be administered in divided doses of two, three, four or more times daily. Furthermore, compositions can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

The dosage regimen utilizing the compositions is selected in accordance with a variety of factors including type, species, age, weight, sex and medical condition of the patient; the severity of the condition to be treated; the route of administration; the renal, hepatic and cardiovascular function of the patient; and the particular composition thereof employed. A physician of ordinary skill can readily determine and prescribe the effective amount of the composition required to prevent, counter or arrest the progress of the condition. Optimal precision in achieving concentrations of composition within the range that yields efficacy without toxicity requires a regimen based on the kinetics of the composition's availability to target sites. This involves a consideration of the distribution, equilibrium, and elimination of a composition.

The inhibitors and activators of potassium channels containing h-erg2 protein will be useful for treating a variety of diseases involving excessive or insufficient potassium channel activity.

Expression of h-erg2 in the human kidney was seen by Northern blot analysis. The kidney is involved in the regulation of salt, water, and pH balance, and is the location of many transporters and channels, many of which are targets for currently available diuretics and other antihypertensive agents (Puschett, 1994, Cardiology 84 Suppl 2:4-13). Expression of h-erg2 was also seen in the prostate. The expression pattern of h-erg2 suggests that inhibitors and activators of potassium channels containing h-erg2 protein are likely to be useful for the treatment of hypo- and hypertension, renal failure, benign prostatic hyperplasia, prostate cancer, and infertility.

The h-erg2 nucleic acids and proteins of the present invention are useful in conjunction with screens designed to identify activators and inhibitors of other ion channels. When screening compounds in order to identify potential pharmaceuticals that specifically interact with a target ion channel, it is necessary to ensure that the compounds identified are as specific as possible for the target ion channel. To do this, it is necessary to screen the compounds against as wide an array as possible of ion channels that are similar to the target ion channel. Thus, in order to find compounds that are potential pharmaceuticals that interact with ion channel A, it is not enough to ensure that the compounds interact with ion channel A (the “plus target”) and produce the desired pharmacological effect through ion channel A. It is also necessary to determine that the compounds do not interact with ion channels B, C, D, etc. (the “minus targets”). The methods used to determine that a compound that is a drug candidate does not interact with minus targets are often referred to as “counterscreens.” In general, as part of a screening program, it is important to use as many minus targets in counterscreens as possible (see Hodgson, 1992, Bio/Technology 10:973-980, at 980). H-erg2 protein, DNA encoding h-erg2 protein, and recombinant cells that have been engineered to express h-erg2 protein have utility in that they can be used as “minus targets” in screening programs designed to identify compounds that specifically interact with other ion channels. For example, Wang et al., 1998, Science 282:1890-1893 have shown that KCNQ2 and KCNQ3 form a heteromeric potassium ion channel know as the “M-channel.” The M-channel is an important target for drug discovery since mutations in KCNQ2 and KCNQ3 are responsible for causing epilepsy (Biervert et al., 1998, Science 279:403-406; Singh et al., 1998, Nature Genet. 18:25-29; Schroeder et al., Nature 1998, 396:687-690). A screening program designed to identify activators or inhibitors of the M-channel would benefit greatly by the use of potassium channels comprising h-erg2 protein as minus targets.

Accordingly, the present invention includes methods for identifying drug candidates that modulate ion channels where the methods encompass using h-erg2 in a counterscreen. Such methods comprise:

(a) determining that a compound is an activator or an inhibitor of an ion channel where the ion channel is not h-erg2; and

(b) determining that the compound is not an activator or an inhibitor of h-erg2.

Of course, h-erg2 may also be valuable in counterscreens where the primary drug target is not an ion channel. Thus, the present invention includes a method for determining that a drug candidate is not an activator or inhibitor of h-erg2 comprising:

(a) selecting a drug target that is not h-erg2;

(b) screening a collection of compounds to identify a compound that is an activator or an inhibitor of the drug target; and

(c) determining that the compound identified in step (b) is not an activator or an inhibitor of h-erg2.

The present invention also includes antibodies to the h-erg2 protein. Such antibodies may be polyclonal antibodies or monoclonal antibodies. The antibodies of the present invention can be raised against the entire h-erg2 protein or against suitable antigenic fragments that are coupled to suitable carriers, e.g., serum albumin or keyhole limpet hemocyanin, by methods well known in the art. Methods of identifying suitable antigenic fragments of a protein are known in the art. See, e.g., Hopp & Woods, 1981, Proc. Natl. Acad. Sci. USA 78:3824-3828; and Jameson & Wolf, 1988, CABIOS (Computer Applications in the Biosciences) 4:181-186.

For the production of polyclonal antibodies, h-erg2 protein or antigenic fragments, coupled to a suitable carrier, are injected on a periodic basis into an appropriate non-human host animal such as, e.g., rabbits, sheep, goats, rats, mice. The animals are bled periodically and sera obtained are tested for the presence of antibodies to the injected h-erg2 protein or antigenic fragment. The injections can be intramuscular, intraperitoneal, subcutaneous, and the like, and can be accompanied with adjuvant.

For the production of monoclonal antibodies, h-erg2 protein or antigenic fragments, coupled to a suitable carrier, are injected into an appropriate non-human host animal as above for the production of polyclonal antibodies. In the case of monoclonal antibodies, the animal is generally a mouse. The animal's spleen cells are then immortalized, often by fusion with a myeloma cell, as described in Kohler & Milstein, 1975, Nature 256:495497. For a fuller description of the production of monoclonal antibodies, see Antibodies: A Laboratory Manual, Harlow & Lane, eds., Cold Spring Harbor Laboratory Press, 1988.

Gene therapy may be used to introduce h-erg2 protein into the cells of target organs. Nucleotides encoding h-erg2 protein can be ligated into viral vectors, which mediate transfer of the nucleotides by infection of recipient cells. Suitable viral vectors include retrovirus, adenovirus, adeno-associated virus, herpes virus, vaccinia virus, lentivirus, and polio virus based vectors. Alternatively, nucleotides encoding h-erg2 protein can be transferred into cells for gene therapy by non-viral techniques including receptor-mediated targeted transfer using ligand-nucleotide conjugates, lipofection, membrane fusion, or direct microinjection. These procedures and variations thereof are suitable for ex vivo as well as in vivo gene therapy. Gene therapy with wild type h-erg2 proteins will be particularly useful for the treatment of diseases where it is beneficial to elevate h-erg2 potassium channel activity. Gene therapy with a dominant negative mutant of h-erg2 protein will be particularly useful for the treatment of diseases where it is beneficial to decrease h-erg potassium channel activity.

The following non-limiting examples are presented to better illustrate the invention.

### EXAMPLE 1

Identification and Cloning of h-erg2 cDNA

The full length h-erg2 cDNA was cloned as three separate but overlapping fragments by PCR-1) the amino terminus to the membrane spanning region S 1, 2) from an overlapping region in S 1 to a region downstream (3′) to the cyclic nucleotide-binding domain (CNBD) and within the EST sequence of H96170, and 3) from a region in the EST to part of the 3′ noncoding sequence. PCR primers were designed from the sequence obtained from genomic DNA database searches. Two identical cDNAs, encoding the amino terminal sequence, were obtained by standard PCR techniques using the following primer pair:

| | |
|---|---|
| 5′ GGAGACGCCGGGAGCCAGTGG 3′ | (SEQ.ID.NO.:15) |
| 5′ GGTATAGCTGCAGGCCCCACG 3′ | (SEQ.ID.NO.:16) |

The S1-CNBD region was similarly cloned by PCR with the following primer pair:

| | |
|---|---|
| 5′ CCAGCTCCACCACGGAGATTG 3′ | (SEQ.ID.NO.:17) |
| 5′ CCAGCTTCAGAGGCCAGCAAT 3′ | (SEQ.ID.NO.:18) |

The C terminal cDNA (post CNBD-3′ untranslated) was cloned by PCR with the following primer pair:

| | |
|---|---|
| 5′ GACGTGACCGGGGGTCTC 3′ | (SEQ.ID.NO.:19) |
| 5′ CTCTCACCTTGCCCACCTTCAG 3′ | (SEQ.ID.NO.:20) |

Three clones were sequenced. One had the 108 base pair insertion shown in SEQ. ID. NO.:5. One clone had a 237 base pair deletion shown in SEQ. ID. NO.:7. The single nucleotide polymorphism at position 2722 was also identified by comparison of the sequences of these cDNAs and the corresponding genomic DNA. The cDNA encoding the full length ORF was then constructed from the 3 partial cDNAs by standard molecular biologic techniques.

### EXAMPLE 2

Analysis of the Expression of h-erg2

Northern Blot Analysis:

Northern blots of poly(A⁺) RNA isolated from human heart, brain, placenta, lung, liver, skeletal muscle, kidney, pancreas, spleen, thymus, prostate, testis, uterus, small intestine, colon, and peripheral blood leukocytes were purchased from Clontech (Palto Alto, Calif.) and probed with a ³²P-labeled probe derived from EST H96170. The probe was constructed by standard PCR techniques using the following primers:

| | |
|---|---|
| 5′ GGACAAAACTCTGGCACCAT 3′ | (SEQ.ID.NO.:21) |
| 5′ GTTCAGGGGAGGGAGGAGAAG 3′ | (SEQ.ID.NO.:22) |

This PCR fragment was purified, and labeled with [³²P]dCTP to a specific activity of >10⁹ cpm/μg DNA by random priming. The hybridization was carried out in a solution containing 5×SSPE, 10× Denhardt's solution, 50% formamide, 100 μg/ml salmon sperm DNA, 2% SDS, and 5×10⁷ cpm ³2P-labeled probe at 42° C. overnight. The blots were washed stepwise in 2×SSC, 0.05% SDS at 42° C. 3 times for 15-20 minutes followed by 1×SSC, 0.05% SDS at 50° C. 3 times for 15-20 minutes. Hybridization was detected by exposure of the blots to Kodak X-ray film.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

What is claimed is:

1. An isolated DNA comprising nucleotides encoding h-erg2.

2. The DNA of claim 1 comprising nucleotides encoding a polypeptide having an amino acid sequence selected from the group consisting of SEQ. ID. NOs.:2, 4, 6, and 8.

3. The DNA of claim 1 comprising a nucleotide sequence selected from the group consisting of: SEQ. ID. NO.:1, SEQ. ID. NO.:3, SEQ. ID. NO.:5, SEQ. ID. NO.:7, positions 57 to 2930 of SEQ. ID. NO.:1, positions 57 to 2930 of SEQ. ID. NO.:3, positions 57 to 3038 of SEQ. ID. NO.:5, and positions 57 to 2693 of SEQ. ID. NO.:7.

4. An isolated DNA that hybridizes under stringent conditions to the DNA of claim 3 and that encodes a protein having substantially the same biological activity as h-erg2.

5. An expression vector comprising the DNA of claim 3.

6. A recombinant host cell comprising the DNA of claim 3.

7. An isolated h-erg2 protein.

8. The protein of claim 7 having an amino acid sequence selected from the group consisting of SEQ. ID. NOs.:2, 4, 6, and 8.

9. The protein of claim 8 containing a single amino acid substitution.

10. The protein of claim 8 containing two or more amino acid substitutions where the amino acid substitutions do not occur in conserved positions.

11. An antibody that binds specifically to a h-erg2 protein.

12. A DNA or RNA oligonucleotide probe comprising at least 10 contiguous nucleotides from SEQ. ID. NO.:1, 3, 5, or 7.

13. A method of identifying substances that bind to potassium channels containing h-erg2 protein comprising:
(a) providing cells expressing a potassium channel containing h-erg2 protein;
(b) exposing the cells to a substance that is not known to bind potassium channels containing h-erg2 protein;
(c) determining the amount of binding of the substance to the cells;
(d) comparing the amount of binding in step (c) to the amount of binding of the substance to control cells where the control cells are substantially identical to the cells of step (a) except that the control cells do not express h-erg2 protein;
where if the amount of binding in step (c) is greater than the amount of binding of the substance to control cells, then the substance binds to potassium channels containing h-erg2 protein.

14. A method of identifying substances that bind potassium channels containing h-erg2 protein comprising:
(a) providing cells expressing potassium channels containing h-erg2 protein;
(b) exposing the cells to a compound that is known to bind to the potassium channels containing h-erg2 protein in the presence and in the absence of a substance not known to bind to potassium channels containing h-erg2 protein;
(c) determining the amount of binding of the compound to the cells in the presence and in the absence of the substance not known to bind to potassium channels containing h-erg2 protein;
where if the amount of binding of the compound in the presence of the substance differs from that in the absence of the substance, then the substance binds potassium channels containing h-erg2 protein.

15. A method of identifying activators or inhibitors of potassium channels containing h-erg2 protein comprising:
(a) recombinantly expressing h-erg2 protein in a host cell so that the recombinantly expressed h-erg2 protein forms potassium channels either by itself or by forming heteromers with other potassium channel subunit proteins;
(b) measuring the biological activity of the potassium channels formed in step (a) in the presence and in the absence of a substance suspected of being an activator or an inhibitor of potassium channels containing h-erg2 protein;
where a change in the biological activity of the potassium channels formed in step (a) in the presence as compared to the absence of the substance indicates that the substance is an activator or an inhibitor of potassium channels containing h-erg2 protein.


SEQUENCE LISTING (12 sequences)

SEQ ID NO: 1 - DNA, 2983 bp, Human
ggagacgccgggagccagtggcgcctgtggctccgggcaggggccgcggccgaaagatgccggtccgcaggggccacgtcgctccccaaaacacttacctggacaccatcatccgcaagttcgagggccaaagtcggaagttcctgattgccaatgctcagatggagaactgcgccatcatttactgcaacgacggcttctgcgaactcttcggctactcccgagtggaggtgatgcagcaaccctgcacctgcgacttcctcacaggccccaacacaccaagcagcgccgtgtcccgcctagcgcaggccctgctgggggctgaggagtgcaaggtggacatcctctactaccgcaaggatgcctccagcttccgctgcctggtagatgtggtgcccgtgaagaacgaggacggggctgtcatcatgttcattctcaacttcgaggacctggcccagctcctggccaagtgcagcagccgcagcttgtcccagcgcctgttgtcccagagcttcctgggctccgagggctctcatggcaggccaggcggaccagggccaggcacaggcaggggcaagtacaggaccatcagccagatcccacagttcacgctcaacttcgtggagttcaacttggagaagcaccgctccagctccaccacggagattgagatcatcgcgccccataaggtggtggagcggacacagaacgtcactgagaaggtcacccaggtcctgtccctgggcgcggatgtgctgccggagtacaagctgcaggcgccgcgcatccaccgctggaccatcctgcactacagccccttcaaggccgtgtgggactggctcatcctgctgctggtcatctacacggctgtcttcacgccctactcagccgccttcctgctcagcgaccaggacgaatcacggcgtggggcctgcagctatacctgcagtcccctcactgtggtggatctcatcgtggacatcatgttcgtcgtggacatcgtcatcaacttccgcaccacctatgtcaacaccaatgatgaggtggtcagccacccccgccgcatcgctgtccactacttcaagggctggttcctcattgacatggtggccgccatccctttcgacctcctgatcttccgcactggctccgatgagaccacaaccctgattgggctattgaagacagcgcggctgctgcggctggtgcgcgtagcacggaagctggaccgctactctgagtatggggcggctgtgctcttcttgctcatgtgcaccttcgcgctcatagcgcactggctggcctgcatctggtacgccatcggcaatgtggagcggccctacctagaacacaagatcggctggctggacagcctgggtgtgcagcttggcaagcgctacaacggcagcgacccagcctcgggcccctcggtgcaggacaagtatgtcacagccctctacttcaccttcagcagcctcaccagcgtgggcttcggcaatgtctcgcccaacaccaactccgagaaggtcttctccatctgcgtcatgctcatcggttccctgatgtacgccagcatcttcgggaacgtgtccgcgatcatccagcgcctgtactcgggcaccgcgcgctaccacacgcagatgctgcgtgtcaaggagttcatccgcttccaccagatccccaacccactgcgccagcgcctggaggagtatttccagcacgcctggtcctacaccaatggcattgacatgaacgcggtgctgaagggcttccccgagtgcctgcaggctgacatctgcctgcacctgcaccgcgcactgctgcagcactgcccagctttcagcggcgccggcaagggctgcctgcgcgcgctagccgtcaagttcaagaccacccacgcgccgcctggggacacgctggtgcacctcggcgacgtgctctccaccctctacttcatctcccgaggctccatcgagatcctgcgcgacgacgtggtcgtggccatcctaggaaagaatgacatctttggggaacccgtcagcctccatgcccagccaggcaagtccagtgcagacgtgcgggctctgacctactgcgacctgcacaagatccagcgggcagatctgctggaggtgctggacatgtacccggcctttgcggagagcttctggagtaagctggaggtcaccttcaacctgcgggacgcagccgggggtctccactcatccccccgacaggctcctggcagccaagaccaccaaggtttctttctcagtgacaaccagtcagatgcagcccctcccctgagcatctcagatgcatctggcctctggcctgagctactgcaggaaatgcccccaaggcacagcccccaaagccctcaggaagacccagattgctggcctctgaagctgggctccaggctagagcagctccaggcccagatgaacaggctggagtcccgcgtgtcctcagacctcagccgcatcttgcagctcctccagaagcccatgccccagggccacgccagctacattctggaagcccctgcctccaatgacctggccttggttcctatagcctcggagacgacgagtccagggcccaggctgccccagggctttctgcctcctgcacagaccccaagctatggggacttggatgactgtagtccaaagcacaggaactcctcccccaggatgcctcacctggctgtggcaatggacaaaactctggcaccatcctcagaacaggaacagcctgaggggctctggccacccctagcctcacctctacatcccctggaagtacaaggactcatctgtggtccctgcttctcctccctccctgaacaccttggctctgttcccaagcagctggacttccagagacatggctcagatcctggatttgcagggagttggggccactgaactccaagataaagacaccatgaggggactgaaggtgggcaaggtgagag

SEQ ID NO: 2 - protein, 958 aa, Human
MPVRRGHVAPQNTYLDTIIRKFEGQSRKFLIANAQMENCAIIYCNDGFCELFGYSRVEVMQQPCTCDFLTGPNTPSSAVSRLAQALLGAEECKVDILYYRKDASSFRCLVDVVPVKNEDGAVIMFILNFEDLAQLLAKCSSRSLSQRLLSQSFLGSEGSHGRPGGPGPGTGRGKYRTISQIPQFTLNFVEFNLEKHRSSSTTEIEIIAPHKVVERTQNVTEKVTQVLSLGADVLPEYKLQAPRIHRWTILHYSPFKAVWDWLILLLVIYTAVFTPYSAAFLLSDQDESRRGACSYTCSPLTVVDLIVDIMFVVDIVINFRTTYVNTNDEVVSHPRRIAVHYFKGWFLIDMVAAIPFDLLIFRTGSDETTTLIGLLKTARLLRLVRVARKLDRYSEYGAAVLFLLMCTFALIAHWLACIWYAIGNVERPYLEHKIGWLDSLGVQLGKRYNGSDPASGPSVQDKYVTALYFTFSSLTSVGFGNVSPNTNSEKVFSICVMLIGSLMYASIFGNVSAIIQRLYSGTARYHTQMLRVKEFIRFHQIPNPLRQRLEEYFQHAWSYTNGIDMNAVLKGFPECLQADICLHLHRALLQHCPAFSGAGKGCLRALAVKFKTTHAPPGDTLVHLGDVLSTLYFISRGSIEILRDDVVVAILGKNDIFGEPVSLHAQPGKSSADVRALTYCDLHKIQRADLLEVLDMYPAFAESFWSKLEVTFNLRDAAGGLHSSPRQAPGSQDHQGFFLSDNQSDAAPPLSISDASGLWPELLQEMPPRHSPQSPQEDPDCWPLKLGSRLEQLQAQMNRLESRVSSDLSRILQLLQKPMPQGHASYILEAPASNDLALVPIASETTSPGPRLPQGFLPPAQTPSYGDLDDCSPKHRNSSPRMPHLAVAMDKTLAPSSEQEQPEGLWPPLASPLHPLEVQGLICGPCFSSLPEHLGSVPKQLDFQRHGSDPGFAGSWGH

SEQ ID NO: 3 - DNA, 2983 bp, Human
ggagacgccgggagccagtggcgcctgtggctccgggcaggggccgcggccgaaagatgccggtccgcaggggccacgtcgctccccaaaacacttacctggacaccatcatccgcaagttcgagggccaaagtcggaagttcctgattgccaatgctcagatggagaactgcgccatcatttactgcaacgacggcttctgcgaactcttcggctactcccgagtggaggtgatgcagcaaccctgcacctgcgacttcctcacaggccccaacacaccaagcagcgccgtgtcccgcctagcgcaggccctgctgggggctgaggagtgcaaggtggacatcctctactaccgcaaggatgcctccagcttccgctgcctggtagatgtggtgcccgtgaagaacgaggacggggctgtcatcatgttcattctcaacttcgaggacctggcccagctcctggccaagtgcagcagccgcagcttgtcccagcgcctgttgtcccagagcttcctgggctccgagggctctcatggcaggccaggcggaccagggccaggcacaggcaggggcaagtacaggaccatcagccagatcccacagttcacgctcaacttcgtggagttcaacttggagaagcaccgctccagctccaccacggagattgagatcatcgcgccccataaggtggtggagcggacacagaacgtcactgagaaggtcacccaggtcctgtccctgggcgcggatgtgctgccggagtacaagctgcaggcgccgcgcatccaccgctggaccatcctgcactacagccccttcaaggccgtgtgggactggctcatcctgctgctggtcatctacacggctgtcttcacgccctactcagccgccttcctgctcagcgaccaggacgaatcacggcgtggggcctgcagctatacctgcagtcccctcactgtggtggatctcatcgtggacatcatgttcgtcgtggacatcgtcatcaacttccgcaccacctatgtcaacaccaatgatgaggtggtcagccacccccgccgcatcgctgtccactacttcaagggctggttcctcattgacatggtggccgccatccctttcgacctcctgatcttccgcactggctccgatgagaccacaaccctgattgggctattgaagacagcgcggctgctgcggctggtgcgcgtagcacggaagctggaccgctactctgagtatggggcggctgtgctcttcttgctcatgtgcaccttcgcgctcatagcgcactggctggcctgcatctggtacgccatcggcaatgtggagcggccctacctagaacacaagatcggctggctggacagcctgggtgtgcagcttggcaagcgctacaacggcagcgacccagcctcgggcccctcggtgcaggacaagtatgtcacagccctctacttcaccttcagcagcctcaccagcgtgggcttcggcaatgtctcgcccaacaccaactccgagaaggtcttctccatctgcgtcatgctcatcggttccctgatgtacgccagcatcttcgggaacgtgtccgcgatcatccagcgcctgtactcgggcaccgcgcgctaccacacgcagatgctgcgtgtcaaggagttcatccgcttccaccagatccccaacccactgcgccagcgcctggaggagtatttccagcacgcctggtcctacaccaatggcattgacatgaacgcggtgctgaagggcttccccgagtgcctgcaggctgacatctgcctgcacctgcaccgcgcactgctgcagcactgcccagctttcagcggcgccggcaagggctgcctgcgcgcgctagccgtcaagttcaagaccacccacgcgccgcctggggacacgctggtgcacctcggcgacgtgctctccaccctctacttcatctcccgaggctccatcgagatcctgcgcgacgacgtggtcgtggccatcctaggaaagaatgacatctttggggaacccgtcagcctccatgcccagccaggcaagtccagtgcagacgtgcgggctctgacctactgcgacctgcacaagatccagcgggcagatctgctggaggtgctggacatgtacccggcctttgcggagagcttctggagtaagctggaggtcaccttcaacctgcgggacgcagccgggggtctccactcatccccccgacaggctcctggcagccaagaccaccaaggtttctttctcagtgacaaccagtcagatgcagcccctcccctgagcatctcagatgcatctggcctctggcctgagctactgcaggaaatgcccccaaggcacagcccccaaagccctcaggaagacccagattgctggcctctgaagctgggctccaggctagagcagctccaggcccagatgaacaggctggagtcccgcgtgtcctcagacctcagccgcatcttgcagctcctccagaagcccatgccccagggccacgccagctacattctggaagcccctgcctccaatgacctggccttggttcctatagcctcggagacgacgagtccagggcccaggctgccccagggctttctgcctcctgcacagaccccaagctatggggacttggatgactgtagtccaaagcacaggaactcctcccccaggatgcctcacctggctgtggcaacggacaaaactctggcaccatcctcagaacaggaacagcctgaggggctctggccacccctagcctcacctctacatcccctggaagtacaaggactcatctgtggtccctgcttctcctccctccctgaacaccttggctctgttcccaagcagctggacttccagagacatggctcagatcctggatttgcagggagttggggccactgaactccaagataaagacaccatgaggggactgaaggtgggcaaggtgagag

SEQ ID NO: 4 - protein, 958 aa, Human
MPVRRGHVAPQNTYLDTIIRKFEGQSRKFLIANAQMENCAIIYCNDGFCELFGYSRVEVMQQPCTCDFLTGPNTPSSAVSRLAQALLGAEECKVDILYYRKDASSFRCLVDVVPVKNEDGAVIMFILNFEDLAQLLAKCSSRSLSQRLLSQSFLGSEGSHGRPGGPGPGTGRGKYRTISQIPQFTLNFVEFNLEKHRSSSTTEIEIIAPHKVVERTQNVTEKVTQVLSLGADVLPEYKLQAPRIHRWTILHYSPFKAVWDWLILLLVIYTAVFTPYSAAFLLSDQDESRRGACSYTCSPLTVVDLIVDIMFVVDIVINFRTTYVNTNDEVVSHPRRIAVHYFKGWFLIDMVAAIPFDLLIFRTGSDETTTLIGLLKTARLLRLVRVARKLDRYSEYGAAVLFLLMCTFALIAHWLACIWYAIGNVERPYLEHKIGWLDSLGVQLGKRYNGSDPASGPSVQDKYVTALYFTFSSLTSVGFGNVSPNTNSEKVFSICVMLIGSLMYASIFGNVSAIIQRLYSGTARYHTQMLRVKEFIRFHQIPNPLRQRLEEYFQHAWSYTNGIDMNAVLKGFPECLQADICLHLHRALLQHCPAFSGAGKGCLRALAVKFKTTHAPPGDTLVHLGDVLSTLYFISRGSIEILRDDVVVAILGKNDIFGEPVSLHAQPGKSSADVRALTYCDLHKIQRADLLEVLDMYPAFAESFWSKLEVTFNLRDAAGGLHSSPRQAPGSQDHQGFFLSDNQSDAAPPLSISDASGLWPELLQEMPPRHSPQSPQEDPDCWPLKLGSRLEQLQAQMNRLESRVSSDLSRILQLLQKPMPQGHASYILEAPASNDLALVPIASETTSPGPRLPQGFLPPAQTPSYGDLDDCSPKHRNSSPRMPHLAVATDKTLAPSSEQEQPEGLWPPLASPLHPLEVQGLICGPCFSSLPEHLGSVPKQLDFQRHGSDPGFAGSWGH

SEQ ID NO: 5 - DNA, 3091 bp, Human
ggagacgccgggagccagtggcgcctgtggctccgggcaggggccgcggccgaaagatgccggtccgcaggggccacgtcgctccccaaaacacttacctggacaccatcatccgcaagttcgagggccaaagtcggaagttcctgattgccaatgctcagatggagaactgcgccatcatttactgcaacgacggcttctgcgaactcttcggctactcccgagtggaggtgatgcagcaaccctgcacctgcgacttcctcacaggccccaacacaccaagcagcgccgtgtcccgcctagcgcaggccctgctgggggctgaggagtgcaaggtggacatcctctactaccgcaaggatgcctccagcttccgctgcctggtagatgtggtgcccgtgaagaacgaggacggggctgtcatcatgttcattctcaacttcgaggacctggcccagctcctggccaagtgcagcagccgcagcttgtcccagcgcctgttgtcccagagcttcctgggctccgagggctctcatggcaggccaggcggaccagggccaggcacaggcaggggcaagtacaggaccatcagccagatcccacagttcacgctcaacttcgtggagttcaacttggagaagcaccgctccagctccaccacggagattgagatcatcgcgccccataaggtggtggagcggacacagaacgtcactgagaaggtcacccaggtcctgtccctgggcgcggatgtgctgccggagtacaagctgcaggcgccgcgcatccaccgctggaccatcctgcactacagccccttcaaggccgtgtgggactggctcatcctgctgctggtcatctacacggctgtcttcacgccctactcagccgccttcctgctcagcgaccaggacgaatcacggcgtggggcctgcagctatacctgcagtcccctcactgtggtggatctcatcgtggacatcatgttcgtcgtggacatcgtcatcaacttccgcaccacctatgtcaacaccaatgatgaggtggtcagccacccccgccgcatcgctgtccactacttcaagggctggttcctcattgacatggtggccgccatccctttcgacctcctgatcttccgcactggctccgatgagaccacaaccctgattgggctattgaagacagcgcggctgctgcggctggtgcgcgtagcacggaagctggaccgctactctgagtatggggcggctgtgctcttcttgctcatgtgcaccttcgcgctcatagcgcactggctggcctgcatctggtacgccatcggcaatgtggagcggccctacctagaacacaagatcggctggctggacagcctgggtgtgcagcttggcaagcgctacaacggcagcgacccagcctcgggcccctcggtgcaggacaagtatgtcacagccctctacttcaccttcagcagcctcaccagcgtgggcttcggcaatgtctcgcccaacaccaactccgagaaggtcttctccatctgcgtcatgctcatcggttccctgatgtacgccagcatcttcgggaacgtgtccgcgatcatccagcgcctgtactcgggcaccgcgcgctaccacacgcagatgctgcgtgtcaaggagttcatccgcttccaccagatccccaacccactgcgccagcgcctggaggagtatttccagcacgcctggtcctacaccaatggcattgacatgaacgcggtgctgaagggcttccccgagtgcctgcaggctgacatctgcctgcacctgcaccgcgcactgctgcagcactgcccagctttcagcggcgccggcaagggctgcctgcgcgcgctagccgtcaagttcaagaccacccacgcgccgcctggggacacgctggtgcacctcggcgacgtgctctccaccctctacttcatctcccgaggctccatcgagatcctgcgcgacgacgtggtcgtggccatcctaggaaagaatgacatctttggggaacccgtcagcctccatgcccagccaggcaagtccagtgcagacgtgcgggctctgacctactgcgacctgcacaagatccagcgggcagatctgctggaggtgctggacatgtacccggcctttgcggagagcttctggagtaagctggaggtcaccttcaacctgcgggacgcagccgggggtctccactcatccccccgacaggctcctggcagccaagaccaccaaggtttctttctcagtgacaaccagtcagggtctccccatgagctggggccccagttcccctctaaaggctacagcctcctgggtcctgggagccagaactccatgggggcaggaccttgtgctccagggcacccagatgcagcccctcccctgagcatctcagatgcatctggcctctggcctgagctactgcaggaaatgcccccaaggcacagcccccaaagccctcaggaagacccagattgctggcctctgaagctgggctccaggctagagcagctccaggcccagatgaacaggctggagtcccgcgtgtcctcagacctcagccgcatcttgcagctcctccagaagcccatgccccagggccacgccagctacattctggaagcccctgcctccaatgacctggccttggttcctatagcctcggagacgacgagtccagggcccaggctgccccagggctttctgcctcctgcacagaccccaagctatggggacttggatgactgtagtccaaagcacaggaactcctcccccaggatgcctcacctggctgtggcaatggacaaaactctggcaccatcctcagaacaggaacagcctgaggggctctggccacccctagcctcacctctacatcccctggaagtacaaggactcatctgtggtccctgcttctcctccctccctgaacaccttggctctgttcccaagcagctggacttccagagacatggctcagatcctggatttgcagggagttggggccactgaactccaagataaagacaccatgaggggactgaaggtgggcaaggtgagag

SEQ ID NO: 6 - protein, 994 aa, Human
MPVRRGHVAPQNTYLDTIIRKFEGQSRKFLIANAQMENCAIIYCNDGFCELFGYSRVEVMQQPCTCDFLTGPNTPSSAVSRLAQALLGAEECKVDILYYRKDASSFRCLVDVVPVKNEDGAVIMFILNFEDLAQLLAKCSSRSLSQRLLSQSFLGSEGSHGRPGGPGPGTGRGKYRTISQIPQFTLNFVEFNLEKHRSSSTTEIEIIAPHKVVERTQNVTEKVTQVLSLGADVLPEYKLQAPRIHRWTILHYSPFKAVWDWLILLLVIYTAVFTPYSAAFLLSDQDESRRGACSYTCSPLTVVDLIVDIMFVVDIVINFRTTYVNTNDEVVSHPRRIAVHYFKGWFLIDMVAAIPFDLLIFRTGSDETTTLIGLLKTARLLRLVRVARKLDRYSEYGAAVLFLLMCTFALIAHWLACIWYAIGNVERPYLEHKIGWLDSLGVQLGKRYNGSDPASGPSVQDKYVTALYFTFSSLTSVGFGNVSPNTNSEKVFSICVMLIGSLMYASIFGNVSAIIQRLYSGTARYHTQMLRVKEFIRFHQIPNPLRQRLEEYFQHAWSYTNGIDMNAVLKGFPECLQADICLHLHRALLQHCPAFSGAGKGCLRALAVKFKTTHAPPGDTLVHLGDVLSTLYFISRGSIEILRDDVVVAILGKNDIFGEPVSLHAQPGKSSADVRALTYCDLHKIQRADLLEVLDMYPAFAESFWSKLEVTFNLRDAAGGLHSSPRQAPGSQDHQGFFLSDNQSGSPHELGPQFPSKGYSLLGPGSQNSMGAGPCAPGHPDAAPPLSISDASGLWPELLQEMPPRHSPQSPQEDPDCWPLKLGSRLEQLQAQMNRLESRVSSDLSRILQLLQKPMPQGHASYILEAPASNDLALVPIASETTSPGPRLPQGFLPPAQTPSYGDLDDCSPKHRNSSPRMPHLAVAMDKTLAPSSEQEQPEGLWPPLASPLHPLEVQGLICGPCFSSLPEHLGSVPKQLDFQRHGSDPGFAGSWGH

SEQ ID NO: 7 - DNA, 2746 bp, Human
ggagacgccgggagccagtggcgcctgtggctccgggcaggggccgcggccgaaagatgccggtccgcaggggccacgtcgctccccaaaacacttacctggacaccatcatccgcaagttcgagggccaaagtcggaagttcctgattgccaatgctcagatggagaactgcgccatcatttactgcaacgacggcttctgcgaactcttcggctactcccgagtggaggtgatgcagcaaccctgcacctgcgacttcctcacaggccccaacacaccaagcagcgccgtgtcccgcctagcgcaggccctgctgggggctgaggagtgcaaggtggacatcctctactaccgcaaggatgcctccagcttccgctgcctggtagatgtggtgcccgtgaagaacgaggacggggctgtcatcatgttcattctcaacttcgaggacctggcccagctcctggccaagtgcagcagccgcagcttgtcccagcgcctgttgtcccagagcttcctgggctccgagggctctcatggcaggccaggcggaccagggccaggcacaggcaggggcaagtacaggaccatcagccagatcccacagttcacgctcaacttcgtggagttcaacttggagaagcaccgctccagctccaccacggagattgagatcatcgcgccccataaggtggtggagcggacacagaacgtcactgagaaggtcacccaggtcctgtccctgggcgcggatgtgctgccggagtacaagctgcaggcgccgcgcatccaccgctggaccatcctgcactacagccccttcaaggccgtgtgggactggctcatcctgctgctggtcatctacacggctgtcttcacgccctactcagccgccttcctgctcagcgaccaggacgaatcacggcgtggggcctgcagctatacctgcagtcccctcactgtggtggatctcatcgtggacatcatgttcgtcgtggacatcgtcatcaacttccgcaccacctatgtcaacaccaatgatgaggtggtcagccacccccgccgcatcgctgtccactacttcaagggctggttcctcattgacatggtggccgccatccctttcgacctcctgatcttccgcactggctccgatgagaccacaaccctgattgggctattgaagacagcgcggctgctgcggctggtgcgcgtagcacggaagctggaccgctactctgagtatggggcggctgtgctcttcttgctcatgtgcaccttcgcgctcatagcgcactggctggcctgcatctggtacgccatcggcaatgtggagcggccctacctagaacacaagatcggctggctggacagcctgggtgtgcagcttggcaagcgctacaacggcagcgacccagcctcgggcccctcggtgcaggacaagtatgtcacagccctctacttcaccttcagcagcctcaccagcgtgggcttcggcaatgtctcgcccaacaccaactccgagaaggtcttctccatctgcgtcatgctcatcggttccctgatgtacgccagcatcttcgggaacgtgtccgcgatcatccagcgcctgtactcgggcaccgcgcgctaccacacgcagatgctgcgtgtcaaggagttcatccgcttccaccagatccccaacccactgcgccagcgcctggaggagtatttccagcacgcctggtcctacaccaatggcattgacatgaacgcggtgctgaagggcttccccgagtgcctgcaggctgacatctgcctgcacctgcaccgcgcactgctgcagcactgcccagctttcagcggcgccggcaagggctgcctgcgcgcgctagccgtcaagttcaagaccacccacgcgccgcctggggacacgctggtgcacctcggcgacgtgctctccaccctctacttcatctcccgaggctccatcgagatcctgcgcgacgacgtggtcgtggccatcctaggaaagaatgacatctttggggaacccgtcagcctccatgcccagccaggcaagtccagtgcagacgtgcgggctctgacctactgcgacctgcacaagatccagcgggcagatctgctggaggtgctggacatgtacccggcctttgcggagagcttctggagtaagctggaggtcaccttcaacctgcgggacgcagccgggggtctccactcatccccccgacaggctcctggcagccaagaccaccaaggtttctttctcagtgacaaccagtcagatgcagcccctcccctgagcatctcagatgcatctggcctctggcctgagctactgcaggaaatgcccccaaggcacagcccccaaagccctcaggaagacccagattgctggcctctgaagctgggctccaggctagagcagctccaggcccagatgaacaggctggagtcccgcgtgtcctcagacctcagccgcatcttgcagctcctccagaagcccatgccccagggccacgccagctacattctggaagcccctgcctccaatgacctggccttggttcctatagcctcggagacgacgagtccagggcccaggctgccccagggctttctgcctcctgcacagctggacttccagagacatggctcagatcctggatttgcagggagttggggccactgaactccaagataaagacaccatgaggggactgaaggtgggcaaggtgagag

SEQ ID NO: 8 - protein, 879 aa, Human
MPVRRGHVAPQNTYLDTIIRKFEGQSRKFLIANAQMENCAIIYCNDGFCELFGYSRVEVMQQPCTCDFLTGPNTPSSAVSRLAQALLGAEECKVDILYYRKDASSFRCLVDVVPVKNEDGAVIMFILNFEDLAQLLAKCSSRSLSQRLLSQSFLGSEGSHGRPGGPGPGTGRGKYRTISQIPQFTLNFVEFNLEKHRSSSTTEIEIIAPHKVVERTQNVTEKVTQVLSLGADVLPEYKLQAPRIHRWTILHYSPFKAVWDWLILLLVIYTAVFTPYSAAFLLSDQDESRRGACSYTCSPLTVVDLIVDIMFVVDIVINFRTTYVNTNDEVVSHPRRIAVHYFKGWFLIDMVAAIPFDLLIFRTGSDETTTLIGLLKTARLLRLVRVARKLDRYSEYGAAVLFLLMCTFALIAHWLACIWYAIGNVERPYLEHKIGWLDSLGVQLGKRYNGSDPASGPSVQDKYVTALYFTFSSLTSVGFGNVSPNTNSEKVFSICVMLIGSLMYASIFGNVSAIIQRLYSGTARYHTQMLRVKEFIRFHQIPNPLRQRLEEYFQHAWSYTNGIDMNAVLKGFPECLQADICLHLHRALLQHCPAFSGAGKGCLRALAVKFKTTHAPPGDTLVHLGDVLSTLYFISRGSIEILRDDVVVAILGKNDIFGEPVSLHAQPGKSSADVRALTYCDLHKIQRADLLEVLDMYPAFAESFWSKLEVTFNLRDAAGGLHSSPRQAPGSQDHQGFFLSDNQSDAAPPLSISDASGLWPELLQEMPPRHSPQSPQEDPDCWPLKLGSRLEQLQAQMNRLESRVSSDLSRILQLLQKPMPQGHASYILEAPASNDLALVPIASETTSPGPRLPQGFLPPAQLDFQRHGSDPGFAGSWGH

SEQ ID NO: 9 - protein, 936 aa, Human
  VARIANT 839: Xaa = Any Amino Acid
MPVRRGHVAPQNTYLDTIIRKFEGQSRKFLIANAQMENCAIIYCNDGFCELFGYSRVEVMQQPCTCDFLTGPNTPSSAVSRLAQALLGAEECKVDILYYRKDASSFRCLVDVVPVKNEDGAVIMFILNFEDLAQLLAKCSSRSLSQRLLSQSFLGSEGSHGRPGGPGPGTGRGKYRTISQIPQFTLNFVEFNLEKHRSSSTTEIEIIAPHKVVERTQNVTEKVTQVLSLGADVLPEYKLQAPRIHRWTILHYSPFKAVWDWLILLLVIYTAVFTPYSAAFLLSDQDESRRGTCSYTCSPLTVVDLIVDIMFVVDIVINFRTTYVNTNDEVVSHPRRIAVHYFKGWFLIDMVAAIPFDLLIFRTGSDETTTLIGLLKTARLLRLVRVARKLDRYSEYGAAVLFLLMCTFPLIAHWLACIWYAIGNVERPYLEHKIGWLDSLGVQLGKRYNGSDPASGPSVQDKYVTALYFTFSSLTSVGFGNVSPNTNSEKVFSICVMLIGSLMYASIFGNVSAIIQRLYSGTARYHTQMLRVKEFIRFHQIPNPLRQRLEEYFQHAWSYTNGIDMNAVLKGFPECLQADICLHLHRALLQHCPAFSGAGKGCLRALAVKFKTTHAPPGDTLVHLGDVLSTLYFISRGSIEILRDDVVVAILGKNDIFGEPVSLHAQPGKSSADVRALTYCDLHKIQRADLLEVLDMYPAFAESFWSKLEVTFNLRDVTGGLHSSPRQAPGSQDHQGFFLSDNQSDAAPPLSISDASGSGLSYCRKCPQSHSPQSPQEDPDCWPLKLGSRLEQLQAQMNRLESRVSSDLSRILQLLQKPMPQGHASYILEAPASNDLALXTPSYGDLDDCSPKHRNSSPRMPHLAVAMDKTLAPSSEQEQPEGLWPPLASPLHPLEVQGLICGPCFSSLPEHLGSVPKQLDFQRHGSDPGFAGSWGH

SEQ ID NO: 10 - protein, 950 aa, Rat
MPVRRGHVAPQNTYLDTIIRKFEGQSRKFLIANAQMENCAIIYCNDGFCELFGYSRVEVMQRPCTCDFLTGPNTPSSAVSRLAQALLGAEECKVDILYYRKDASSFRCLVDVVPVKNEDGAVIMFILNFEDLAQLLAKSSSRSLTQRLLSHSFLGSEGSHSRPSGQGPGPGRGKYRTVSQIPQFTLNFVEFNLEKHRSGSTTEIEIIAPHKVVERTQNVTEKVTQVLSLGADVLPEYKLQAPRIHRGTILHYSPFKAVWDWLILLLVIYTAVFTPYSAAFLLSDQDESQRGTCGYTCSPLTVVDLIVDIMFVVDIVINFRTTYVNTNDEVVSHPRRIAVHYFKGWFLIDMVAAIPFDLLIFRTGSDETTTLIGLLKTARLLRLVRVARKLDRYSEYGAAVLFLLMCTFALIAHWLACIWYAIGNVERPYLEPKIGWLDSLGAQLGKQYNGSDPASGPSVQDKYVTALYFTFSSLTSVGFGNVSPNTNSEKVFSICVMLIGSLMYASIFGNVSAIIQRLYSGTARYHTQMLRVKEFIRFHQIPNPLRQRLEEYFQHAWSYTNGIDMNAVLKGFPECLQADICLHLHRALLQHCPAFRGASKGCLRALAVKFKTTHAPPGDTLVHLGDVLSTLYFISRGSIEILRDDVVVAILGKNDIFGEPASLHARPGKSSADVRALTYCDLHKIHRADLLEVLDMYPAFADTFWNKLEVTFNLRDADGGLQSTPRQAPGHQDPQGFFLNDSQSGAAPSLSISDTSALWPELLQQMPPSPPNPRQDLDCWHRELGFKLEQLQAQMNRLESRVSSDLSRILQLLQHPQGRPSYILGASASSDLASFPETSVTRSSESTLLVGHVPSAQTLSYGDLDDHIQTPRNFSPRTPHVAMAMDKTLVPSSEQEQPGGLLSPLASPLRPLEVPGLGGSRFPSLPEHLSSVPKQLEFQRHGSDPGFTRS

SEQ ID NO: 11 - protein, 1159 aa, Human
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 12 - protein, 885 aa, Artificial Sequence
  Synthetic
MPVRRGHVAPQNTYLDTIIRKFEGQSRKFLIANAQMENCAIIYCNDGFCELFGYSRVEVMQRPCTCDFLTGPNTPSSAVSRLAQALLGAEECKVDILYYRKDASSFRCLVDVVPVKNEDGAVIMFILNFEDLAQLLAKSSRSLTQRLLSSFLGSEGSHSRPGGPGGRGKYRTISQIPQFTLNFVEFNLEKHRSSSTTEIEIIAPHKVVERTQNVTEKVTQVLSLGADVLPEYKLQAPRIHRWTILHYSPFKAVWDWLILLLVIYTAVFTPYSAAFLLSDQDESRGTCGYTCSPLTVVDLIVDIMFVVDIVINFRTTYVNTNDEVVSHPRRIAVHYFKGWFLIDMVAAIPFDLLIFRTGSDETTTLIGLLKTARLLRLVRVARKLDRYSEYGAAVLFLLMCTFALIAHWLACIWYAIGNVERPYLEKIGWLDSLGQLGKYNGSDPASGPSVQDKYVTALYFTFSSLTSVGFGNVSPNTNSEKVFSICVMLIGSLMYASIFGNVSAIIQRLYSGTARYHTQMLRVKEFIRFHQIPNPLRQRLEEYFQHAWSYTNGIDMNAVLKGFPECLQADICLHLHRALLQHCPAFRGASKGCLRALAVKFKTTHAPPGDTLVHLGDVLSTLYFISRGSIEILRDDVVVAILGKNDIFGEPVSLHARPGKSSADVRALTYCDLHKIHRADLLEVLDMYPAFADSFWSKLEVTFNLRDGGLSSPRQAPGQDQGFFLSDNQSAAPSLSISDSGLSRQPSPPPDLDCWPLLGSRLEQLQAQMNRLESRVSSDLSRILQLLQKMPQGPSYILASASDLATSYGDLDDPPRNSSPRMPHLAVAMDKTLPSSEQEQPGGLPLASPLPLEVPGLSRFSLPEHLGSVPKQLDFQRHGSDPGFS